(19) United States
(12) Patent Application Publication
(10) Pub. No.: US 2007/0264349 A1
(43) Pub. Date: Nov. 15, 2007

(54) Nano-structured compositions and methods of making and using the same

(75) Inventors: Robert Lee, Malvern, PA (US); Dinesh Shenoy, Malvern, PA (US); D. Craig Wright, Malvern, PA (US)

Correspondence Address: FOLEY AND LARDNER LLP;SUITE 500, 3000 K STREET NW, WASHINGTON, DC, 20007, US

(73) Assignee: Novavax, Inc.

(21) Appl. No.: 11/714,274

(22) Filed: Mar. 6, 2007

Related U.S. Application Data
(60) Provisional application No. 60/779,420, filed on Mar. 7, 2006.
(60) Provisional application No. 60/837,294, filed on Aug. 14, 2006.

(51) Int. Cl. A61K 9/14; A61K 31/19; A61K 31/337; A61K 31/439; A61K 31/495; A61K 31/56; A61K 31/568; A61K 31/57; A61K 31/662; A61K 31/7052; A61K 36/06

(52) U.S. Cl. 424/489; 424/195.15; 514/108; 514/177; 514/178; 514/255.04; 514/282; 514/343; 514/449; 514/45; 514/570

(57) ABSTRACT

The present invention provides a new tri-phasic method for making nanoparticles of poorly soluble active pharmaceutical ingredients.

### CROSS-REFERENCE TO RELATED APPLICATIONS

This application claims priority from U.S. Provisional Application Nos. 60/779,420, filed Mar. 7, 2006, and 60/837,294, filed Aug. 14, 2006. The contents of these applications are incorporated by reference.

### FIELD OF THE INVENTION

The invention is directed to methods for preparing nano-structures of active pharmaceutical ingredients, compositions made by the novel methods, and methods of using the compositions.

### BACKGROUND

A. Pharmaceutical Compositions

Ease of active pharmaceutical ingredient delivery is a key issue facing pharmaceutical companies that develop and attempt to commercialize therapeutic products. An active pharmaceutical ingredient (API) that is readily soluble in water, for example, is not difficult to formulate into a suitable dosage form. However, formulating poorly water-soluble therapeutic drugs into suitable dosage forms poses a significant challenge. This is because the human body is a water based system; thus, as a condition of producing therapeutic activity, a drug must dissolve following administration.

Some poorly water-soluble API are never commercialized because they cannot be effectively solubilized, and therefore fail to exhibit acceptable in vivo therapeutic activity. Alternatively, the quantity of poorly water-soluble API required to be administered to achieve an acceptable level of therapeutic activity may be too great, given the poor water solubility of the agent, and result in unacceptable toxicity. Even if an API is formulated into a liquid, wherein the API is solubilized in a solvent, such dosage forms sometimes perform sub-optimally. For example, such dosage forms may have unpredictable properties or induce undesirable side effects. An example of such a solvent is Cremophor®, which is used to solubilize active agents such as taxol. However, in certain subjects Cremophor® induces severe adverse allergic reaction, which has resulted in death.

Prior art methods exist for enhancing API solubility. For example, the particle size of the API can be reduced, thereby increasing the exposed surface area of the API, resulting in greater water solubility. In addition, it is known that small particles, e.g., a micron or smaller, can more easily traverse the skin boundary than larger particles. One prior method for particle size reduction is wet milling. This method requires grinding of an API with beads made of hard glass, porcelain, ceramic (including zirconium oxide and zirconium silicate), polymeric resin, or other suitable substance in a media in which the API is poorly soluble, such as water. The API is physically converted into smaller particles that remain suspended in the grinding media. The resultant micron- or nanometer-sized API particles can then be isolated from the grinding media by methods such as filtration or centrifugation, and formulated into an appropriate dosage form. See U.S. Pat. No. 5,145,684 for “Surface Modified Drug Nanoparticles;” U.S. Pat. Nos. 5,518,187 and 5,862,999, both for “Method of Grinding Pharmaceutical Substances;” and U.S. Pat. No. 5,718,388, for “Continuous Method of Grinding Pharmaceutical Substances.” The media in which the API is ground typically contains one or more compounds that function as a surface stabilizer for the API. The surface stabilizers adsorb to the surface of the API and act as a steric and/or electrostatic barrier to API particle size growth.

Conventional wet milling techniques therefore produce a “bi-phasic” system in which the stabilized API nanoparticles are suspended in a liquid or aqueous media. However, wet milling of API has drawbacks, principally being the cost of the process. The added cost for formulating a poorly water-soluble API into a nanoparticulate composition utilizing wet milling can be prohibitive. Additionally, wet milling techniques are not well suited for processing amorphous or semi-amorphous API's.

Other known methods of making nanoparticulate active agent compositions include precipitation, homogenization, and super critical fluid methods. Microprecipitation is a method of preparing stable dispersions of poorly soluble API. Such a method comprises dissolving an API in a solvent followed by precipitating the API out of solution. Homogenization is a technique that does not use milling or grinding media. API in a liquid media constitutes a process stream propelled into a process zone, which in a Microfluidizer® (Microfluidics, Inc.) is called the Interaction Chamber. The geometry of the interaction chamber produces powerful forces of sheer, impaction, and cavitation which are responsible for particle size reduction. U.S. Pat. No. 5,510,118 refers to a bi-phasic process using a Microfluidizer® resulting in nanoparticulate active agent particles. Finally, supercritical fluid methods of making nanoparticulate API compositions comprise dissolving an API in a solution. The solution and a supercritical fluid are then co-introduced into a particle formation vessel. The temperature and pressure are controlled, such that dispersion and extraction of the vehicle occur substantially simultaneously by the action of the supercritical fluid. Examples of known supercritical methods of making nanoparticles include International Patent Application No. WO 97/144407 and U.S. Pat. No. 6,406,718.

B. Background Regarding Transdermal Dosage Forms

Problems exist with transdermal applications for small particulate drugs. Small particles of drug typically provide only small amounts of drug and therefore their usefulness can be limited. In addition, not all drugs can be formulated into small particulate drug dosage forms, as typically such dosage forms are only suitable for poorly water-soluble drugs. See e.g., U.S. Pat. No. 5,145,684. However, larger sized particles may have more trouble diffusing across the skin barrier.

Transdermal drug delivery permits controlled release of a drug into a patient without directly invading the patient's body. This painless clinical technique can conveniently and effectively deliver drug doses into and through the patient's skin in a passive and continuous manner over the course of hours, days, or weeks. A transdermal patch can be placed essentially anywhere on the skin, such as under clothing, and is therefore discreet and cosmetically elegant. Its ease of use also increases patient compliance with drug administration. An individual does not have to adhere to a strict oral regimen, for example, and does not have to perform routine injections or travel to a clinic for such treatment. Also, by delivering a drug directly into the blood stream, only a minimum effective amount of a drug is required, which can help reduce potential side effects. Furthermore, by delivering a drug directly into the skin and bloodstream, a transdermally-delivered drug bypasses the gastrointestinal tract, thereby eliminating first-pass liver metabolism, which may reduce or destroy a drug's bioactivity.

Transdermal delivery also creates steady levels of a drug in the bloodstream and helps to improve drug efficacy. Depending on various ingredients that are used to formulate the drug, as well as technical aspects of the patch, such as its design and adhesive qualities, the rate of release of the drug can be precisely manipulated. Accordingly, by applying different types of adhesive patches to the skin, more or less or the same amount of drug is administered to an individual over a recommended course of time. Because of these advantages, a transdermally formulated drug is often perceived as more desirable than traditional drug delivery systems, such as injections and orally-administered tablets. Indeed, the drug industry has created patches for fentanyl, nitroglycerin, estradiol, ethinyl estradiol, norethindrone acetate, testosterone, clonidine, nicotine, lidocaine, prilocalne, oxybutynin, and scopolamine, as well as contraceptive patches containing ethinyl estradiol and norelgestromin. The U.S. transdermal market approached $1.2 billion in 2001 and is anticipated to grow world-wide to $13 billion before 2008 (Cleary, G W, 2004. Transdermal & Transdermal-like Delivery System Opportunities Today & the Future, Drug Delivery Technology, 3(5).).

The terms “transdermal” and “patch” imply a limited type of mechanisms for delivery of a drug into a patient's body. In reality, the landscape concerning the types of transdermal devices useful for transdermal delivery is diverse. There exists, for instance, various patch designs that include, for example, drug-in-adhesive patches, multi-layer-drug-in-adhesive patches, microstructured systems, reservoir dispenser systems, membranes, penetration enhancer technologies, hydrogels, gels, micro-emulsions, and film-forming polymers.

Even though transdermal dosage forms are desirable in terms of patient compliance and other factors, there exists in the art problems with formulating drugs into transdermal dosage forms. For example, at present it is not possible to formulate all drugs, biological compounds, and therapeutic proteins for transdermal delivery. The solubility, physicochemical characteristics, and bioavailability of a drug can greatly influence its ability to be formulated into an appropriate transdermal composition.

Moreover, even if a drug can be formulated into a transdermal dosage form, the skin itself is often a barrier, limiting the number and types of drugs that can passively diffuse from the transdermal device and across the skin. This does not mean that transdermal dosage forms are not adaptable. Indeed, it is possible to forcefully drive drugs across the skin barrier, as opposed to relying on passive diffusion. For instance, techniques that help increase skin permeation include ionotophoresis, which uses low voltage electrical current to drive charges drug particles across the skin, and sonophoresis, which uses low frequency ultrasonic energy for the same purpose. Another relatively new technique comprises the use of microstructured arrays of needles, e.g., microneedles that painlessly create micropores in the skin without bleeding when the patch is applied. The size of the newly-created pores can typically accommodate drugs that cannot be suitably prepared for the more traditional transdermal techniques.

Even with the availability of different devices, a drug may have to be reformulated to increase its suitability for transdermal delivery, regardless of which device appears to be the most effective. Indeed, ease of active pharmaceutical agent delivery is a key issue that faces all pharmaceutical companies that develop and commercialize therapeutic products for transdermal, as well as conventional, administration. An active pharmaceutical ingredient (API) that is readily soluble in water, for example, is not difficult to formulate into a suitable dosage form. However, formulating poorly water-soluble API into suitable dosage forms poses a significant challenge. This is because the human body is a water based system; thus, as a condition of producing therapeutic activity, a drug must dissolve following administration.

Some poorly water-soluble API are never commercialized because the API cannot be effectively solubilized, and therefore fail to exhibit acceptable in vivo therapeutic activity. Alternatively, the quantity of poorly water-soluble API required to be administered to achieve an acceptable level of therapeutic activity may be too great, given the poor water solubility of the agent, and result in unacceptable toxicity. Even if an API is formulated into a liquid, wherein the API is solubilized in a solvent, such dosage forms sometimes perform sub-optimally. For example, such dosage forms may have unpredictable properties or induce undesirable side effects. An example of such a solvent is Cremophor®, which is used to solubilize active agents such as paclitaxel. However, in certain subjects Cremophor® induces severe adverse allergic reaction, which has resulted in death.

There is a need in the art for cost-effective methods of formulating poorly water-soluble and water-soluble API into suitable dosage forms exhibiting optimal in vivo efficacy. The present invention satisfies these needs. In addition, there is a need in the art, therefore, for cost-effective methods of formulating poorly water-soluble and water-soluble API into transdermal delivery dosage forms exhibiting optimal in vivo efficacy. The present invention satisfies these needs.

### SUMMARY

One aspect of the invention is directed to a unique active pharmaceutical ingredient nano-structured formulation, which comprises (1) a micelle component, (2) a hydro-alcoholic component, e.g., a mixture of water and water-miscible solvent, (3) an oil-in-water emulsion droplet component, and (4) a solid particle component. Any or all of these components may comprise a desired active pharmaceutical ingredient. Thus, the active pharmaceutical ingredient may be in solution, as denoted in components 1 to 3, or it may be in precipitated suspension form, as is the case in component 4.

Another aspect of the present invention is directed to a pharmaceutical composition comprising: (1) at least one active pharmaceutical ingredient, (2) at least one solvent, (3) at least one oil, (4) at least one surfactant, and (5) water. The active pharmaceutical ingredient can be present in (a) a solid nanoparticulate state; (b) a solid microparticulate state; (c) solubilized; or (d) any combination thereof. In another embodiment of the invention, the composition is suitable for transdermal delivery. In yet another embodiment of the invention, the composition which is suitable for transdermal delivery provides for a depot effect.

Another aspect of the invention is directed to pharmaceutical compositions of the invention suitable for topical application, such as applications via opthalmic, mucosal, otic, dermal, buccal, inhalation, etc.

In one embodiment, described are pharmaceutical compositions comprising macromolecules, e.g., molecules having a molecular weight of greater than about 500 Da. An example of such a compound is cyclosporine. In yet another embodiment of the invention, such pharmaceutical formulations can be delivered to a subject either topically or transdermally.

In another embodiment of the invention, the biphasic and triphasic pharmaceutical compositions or emulsions are suitable for topical application of hydrophilic drugs, including drugs that are highly soluble on both water and oil. As defined herein, “soluble” drugs have solubility in water or another media of greater than about 10 mg/mL, greater than about 20 mg/mL, or greater than about 30 mg/mL.

In one embodiment, when the composition is applied to the skin, the solubilized form travels across the skin and into deeper dermal layers, such as into the dermis. The other components, such as the micelles, oil fraction, and/or the particulate drug may typically position themselves towards the Stratum corneum of the skin layer. Depending on various physical and chemical properties, certain compounds may position themselves in different layers of epidermis and dermis, while others might permeate directly across the skin.

This composite formulation avoids having to incorporate chemical permeation enhancers that are otherwise necessary to induce transdermal permeation of the active pharmaceutical ingredient.

In one embodiment of the invention, the pharmaceutical compositions of the invention have anti-microbial properties.

In yet another embodiment, the pharmaceutical compositions of the invention comprise an antiviral compound. An example of such an antiviral compound is acyclovir.

In one embodiment of the invention, the pharmaceutical compositions of the invention comprise a compound useful in the relief of symptoms associated with perennial and seasonal allergic rhinitis; vasomotor rhinitis; allergic conjunctivitis; mild, uncomplicated urticaria and angioedema; or the amelioration of allergic reactions to blood or plasma; or dermatographism or as adjunctive therapy in anaphylactic reactions. Examples of such compounds include, but are not limited to, loratidine, desloratidine, and cetirizine.

In one embodiment, the active pharmaceutical ingredient is acyclovir, cyclosporine, naltrexone, alendronic acid, ceterizine, nicotine, testosterone, progesterone, or estradiol.

In another embodiment, the composition comprises globules of oil comprising dissolved active pharmaceutical ingredient. The globules can have a diameter of less than about 2 microns. In other embodiments of the invention, the oil globules can have a smaller diameter. In another embodiment, the oil is soybean oil, squalane, tricaprylin, or mineral oil (light).

In one embodiment, the solvent is an alcohol or N-methylpyrrolidinone.

In another embodiment of the invention, provided are micellar nanoparticle and or microparticle drug compositions which are heat stable and therefore amenable to heat sterilization. Micellar nanoparticles are quite viscous and cannot be readily sterilized using aseptic filtration devices, such as filtration using a 0.2 micron filter. Terminal heat sterilization, however, is a desirable method for sterilizing such pharmaceutical compositions. A problem is that, typically, micellar nanoparticle formulations are not stable at elevated temperatures, e.g., at temperatures above 50° C., and therefore cannot be readily autoclaved. Surprisingly, following heat sterilization, the compositions of the invention retain their chemical stability, physical stability, or a combination of chemical and physical stability.

In another aspect of the invention provided is a method for preparing particles of an active pharmaceutical ingredient, which comprises (a) adding the active pharmaceutical ingredient to a mixture of oil, solvent, stabilizer, and water to form an emulsion base, wherein the active pharmaceutical ingredient is poorly soluble in the oil, solvent, and water, (b) homogenizing the emulsion base, and (c) milling the homogenized mix to form particles of the active pharmaceutical ingredient.

Another aspect of the invention is directed to a method for preparing particles of an active pharmaceutical ingredient (API) comprising: (1) forming an emulsion base by suspending an API in a mixture of (i) non-miscible liquid, (ii) solvent, and (iii) water or buffer, and (2) homogenizing or vigorously stirring the emulsion base, wherein the resultant composition is a mixture of API particles suspended in emulsion droplets and sterically stabilized microcrystalline or nanoparticulate API in the media. In one embodiment, the API has a diameter of less than about 10 microns, less than about 9 microns, less than about 8 microns, less than about 7 microns, less than about 6 microns, less than about 5 microns, less than about 4 microns, less than about 3 microns, less than about 2 microns, or less than about 1 micron in size. In another embodiment, the API is acyclovir, cyclosporine, naltrexone, alendronic acid, cetirizine, nicotine, testosterone, progesterone, or estradiol.

Another aspect of the invention is directed to a method for preparing particles of an active pharmaceutical ingredient (API), comprising (1) dissolving an API in a mixture of (i) oil, (ii) solvent, and (iii) stabilizer to form an emulsion pre-mix, (2) adding water or buffer to the emulsion pre-mix, and (3) homogenizing or vigorously stirring the mixture, whereby the API is precipitated into particles. In one embodiment, the diameter of the API is less than about 10 microns, less than about 9 microns, less than about 8 microns, less than about 7 microns, less than about 6 microns, less than about 5 microns, less than about 4 microns, less than about 3 microns, less than about 2 microns, or less than about 1 micron. In another embodiment, the API is acyclovir, cyclosporine, naltrexone, alendronic acid, cetirizine, nicotine, testosterone, progesterone, or estradiol.

In yet another embodiment, the active pharmaceutical ingredient is selected from the group consisting of, but not limited to fenofibrate, estradiol, alendronic acid, acyclovir, paclitaxel, and cyclosporine. In one embodiment, the oil is selected from the group consisting of, but not limited to, almond oil (sweet), apricot seed oil, borage oil, canola oil, coconut oil, corn oil, cotton seed oil, fish oil, jojoba bean oil, lard oil, linseed oil (boiled), Macadamia nut oil, medium chain triglycerides, mineral oil, olive oil, peanut oil, safflower oil, sesame oil, soybean oil, squalene, sunflower seed oil, tricaprylin (1,2,3-trioctanoyl glycerol), and wheat germ oil. In one embodiment, the solvent is selected from the group consisting of, but not limited to isopropyl myristate, triacetin, N-methylpyrrolidinone, aliphatic and aromatic alcohols, polyethylene glycols, and propylene glycol. Other examples of useful solvents are long-chain alcohols. Ethanol is yet another example of an alcohol that may be used in the present invention.

In yet another embodiment, the stabilizer is selected from the group consisting of, but not limited to, sorbitan esters, glycerol esters, polyethylene glycol esters, block polymers, acrylic polymers (such as Pemulen), ethoxylated fatty esters (such as Cremophor RH-40), ethoxylated alcohols (such as Brij), ethoxylated fatty acids (such as Tween), monoglycerides, silicon based surfactants, and polysorbates. Finally, in a further embodiment, the sorbitan ester stabilizer is Span and Arlacel, wherein the glycerol ester is glycerin monostearate, wherein the polyethylene glycol ester is polyethylene glycol stearate, wherein the block polymer is a Pluronic, wherein the acrylic polymer is Pemulen, wherein the ethoxylated fatty ester is Cremophor RH-40, wherein the ethoxylated alcohol is Brij, and wherein the ethoxylated fatty acid is Tween 20.

In another embodiment, a homogenizing step is performed via a high-pressure system at 1,000 to 40,000 psi.

In one embodiment, the active pharmaceutical ingredient particles, droplets comprising API, or a combination thereof have a mean particle size of less than about 10 microns. In other embodiments of the invention, the active pharmaceutical ingredient particles, droplets comprising API, or a combination thereof have a mean particle size of less then about 9 microns, less than about 8 microns, less than about 7 microns, less than about 6 microns, less than about 5 microns, less than about 4 microns, less than about 3 microns, less than about 2900 nm, less than about 2800 nm, less than about 2700 nm, less than about 2600 nm, less than about 2500 nm, less than about 2400 nm, less than about 2300 nm, less than about 2200 nm, less than about 2100 nm, less than about 2000 nm, less than about 1900 nm, less than about 1800 nm, less than about 1700 nm, less than about 1600 nm, less than about 1500 n, less than about 1400 nm, less than about 1300 nm, less than about 1200 nm, less than about 1500 nm, less than about 1000 nm, less than about 900 nm, less than about 800 nm, less than about 700 nm, less than about 600 nm, less than about 500 nm, less than about 400 nm, less than about 300 nm, less than about 200 nm, or less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In one embodiment, the active pharmaceutical ingredient particles, droplets comprising API, or a combination thereof have a mean particle size of less than about 3 microns in diameter.

Another aspect of the invention is directed to a method for preparing particles of an active pharmaceutical ingredient, comprising (a) adding the active pharmaceutical ingredient to a mixture of oil, solvent, and stabilizer to form an emulsion base, wherein the active pharmaceutical ingredient is soluble in either or both of oil and solvent, but is not soluble in water, (b) adding water to the emulsion base, (c) homogenizing the mixture, and (d) milling the homogenized mix to form particles of the active pharmaceutical ingredient.

In another aspect of the invention, a method for preparing fenofibrate particles is provided, which comprises (a) dissolving a suitable amount of fenofibrate in N-methyl-pyrrolidinone to form a solution, (b) adding medium chain triglyceride to the solution, (c) adding Pluronic dissolved in water to the solution, (c) mixing the solution, and (d) subjecting the solution to high-pressure homogenization to create fenofibrate particles.

Another aspect of the invention is directed to a method of administering an active pharmaceutical ingredient to a subject, comprising applying to the subject a composition comprising: (1) at least one active pharmaceutical ingredient, (2) at least one solvent, (3) at least one oil, (4) at least one surfactant, and (5) water, wherein the active pharmaceutical ingredient is present in both a solid nanoparticulate state and in a soluble state. In one embodiment, the composition is applied as a topical cream onto the skin of the individual. In another embodiment, a transdermal patch comprises the composition and the patch is placed into contact with the skin of the subject.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1: Shows the particle size distribution of raw fenofibrate, with a mean particle size of 57 μm.

FIG. 2: Shows the particle size distribution of fenofibrate following particle size reduction using the method of the invention, with a mean fenofibrate nanoemulsion droplet size of 60 nm (within emulsion droplets), and with 100% of the fenofibrate particles having a size of less than 3 μm prepared.

FIG. 3: Shows the particle size distribution for raw acyclovir, with a mean nanoemulsion droplet acyclovir particle size of 54 μm.

FIG. 4: Shows the particle size distribution for acyclovir following particle size reduction using the method of the invention, with a mean nanoemulsion droplet acyclovir size of 132 nm, and with 100% of the acyclovir particles having a size of less than 3 μm.

FIG. 5: Shows the mean cumulative concentration released (μg/sq·cm) over time for transdermal delivery of two different nanoparticulate acyclovir compositions prepared according to the invention (Compositions IV and VI) as compared to a conventional, non-nanoparticulate commercial cream formulation of the same drug, ZOVIRAX®.

FIG. 6: Shows the results of topical and transdermal delivery of acyclovir. The values in parenthesis indicate flux rate of the drug across cadaver skin (e.g., a natural or artificial membrane).

FIG. 7: Shows the results of in vitro release studies across cadaver skin (e.g., a natural or artificial membrane) to determine the effect of change in solvent on estradiol release. The values in parenthesis indicate flux rate of the drug across the membrane.

FIG. 8: Shows the results of in vitro release studies across cadaver skin (e.g., a natural or artificial membrane) to determine the effect of solid crystalline estradiol. The values in parenthesis indicate flux rate of the drug across the membrane.

FIG. 9: Shows a particle size distribution for acyclovir (formulation comprising N-methylpyrrolidone).

FIG. 10: Shows a particle size distribution for acyclovir (formulation comprising ethanol).

FIG. 11: Shows a particle size distribution for cyclosporine.

FIG. 12: Shows in vivo data demonstrating the effect of a change in oil on estradiol release.

FIG. 13: Shows in vivo data regarding the change in blood levels of cetirizine in rabbits over time.

FIG. 14: Shows in vivo data regarding the change in blood levels of nicotine in rabbits over time.

FIG. 15: Shows the transdermal delivery profile of naltrexone hydrochloride in rabbits.

### DETAILED DESCRIPTION

A. Overview of the Invention

The invention is directed to pharmaceutical dosage forms, such as but not limited to transdermal and topical dosage forms, or transdermal drug delivery systems, comprising an active pharmaceutical ingredient (API) and methods of making and using the same. In one embodiment of the invention, a formulation of an API of the invention may be incorporated into a transdermal drug delivery system, such as a cream, ointment, patch, etc., and applied to a subject's skin to deliver the API locally and systemically.

The compositions of the invention can be formulated into any suitable dosage form. Exemplary pharmaceutical dosage forms include, but are not limited to: (1) dosage forms for administration selected from the group consisting of oral, pulmonary, intravenous, rectal, otic, opthalmic, colonic, parenteral, intracisternal, intravaginal, intraperitoneal, local, buccal, nasal, and topical administration; (2) dosage forms selected from the group consisting of liquid dispersions, gels, aerosols, ointments, creams, tablets, sachets and capsules; (3) dosage forms selected from the group consisting of lyophilized formulations, fast melt formulations, controlled release formulations, delayed release formulations, extended release formulations, pulsatile release formulations, and mixed immediate release and controlled release formulations; or (4) any combination thereof.

One aspect of the present invention is directed to a pharmaceutical composition comprising: (1) at least one active pharmaceutical ingredient, (2) at least one solvent, (3) at least one oil, (4) at least one surfactant, and (5) water. The active pharmaceutical ingredient can be present in (a) a solid nanoparticulate state; (b) a solid microparticulate state; (c) solubilized; or (d) any combination thereof. In another embodiment of the invention, the composition is suitable for transdermal delivery. In yet another embodiment of the invention, the composition which is suitable for transdermal delivery provides for a depot effect.

Another aspect of the invention is directed to pharmaceutical compositions of the invention suitable for topical application, such as applications via opthalmic, mucosal, otic, dermal, buccal, inhalation, etc.

In another embodiment of the invention, the biphasic and triphasic pharmaceutical compositions or emulsions of the invention are suitable for topical application of hydrophilic drugs, including drugs that are highly soluble on both water and oil. As defined herein, “soluble” drugs have a solubility in water or another media of greater than about 10 mg/mL, greater than about 20 mg/mL, or greater than about 30 mg/mL.

In one embodiment of the invention, the pharmaceutical compositions of the invention comprise a compound useful in the relief of symptoms associated with perennial and seasonal allergic rhinitis; vasomotor rhinitis; allergic conjunctivitis; mild, uncomplicated urticaria and angioedema; or the amelioration of allergic reactions to blood or plasma; or dermatographism or as adjunctive therapy in anaphylactic reactions. Examples of such compounds include, but are not limited to, loratidine, desloratidine, and cetirizine.

The composition to be utilized in the pharmaceutical dosage forms can be a tri-phasic composition comprising a lipophilic phase, water or a buffer, and particulate API. The composition may also comprise an oil phase that has at least one oil, at least one solvent, and a surface stabilizer for the API.

The invention encompasses a method of making a tri-phasic composition comprising a lipophilic phase, water or a buffer, and particulate API. The invention also encompasses compositions comprising an oil phase that has at least one oil, at least one solvent, and a surface stabilizer for the API. Two specific methods of making the compositions of the invention are described. In the first method (“Route I”), API is milled in an emulsion base. This method requires that the API is poorly soluble or insoluble in all phases of the oil phase/lipophilic phase and the water or buffer. In the second method (“Route II”), simultaneous milling and precipitation of the API in an emulsion base is observed. The second method requires that the API is soluble or partially soluble in one or more phases of the emulsion base; e.g., that the API is soluble in an oil, solvent, or water or buffer.

One benefit of the methods of the invention as compared to prior art methods, such as wet milling, is that the methods are applicable to water-soluble API as well as poorly water soluble API. Another benefit of the methods of the invention is that it does not require grinding media or specialized grinding process or equipment. The use of such grinding media can add cost and complexity to a particle size reduction process for an API. Additionally, unlike wet milling technologies, the methods of the invention can accommodate amorphous or semi-amorphous API's.

For Route I, an API is first suspended in a mixture of a non-miscible liquid, such as an oil, solvent, water or buffer, to form an emulsion base, followed by homogenization or vigorous stirring of the emulsion base. Nanoparticles can be produced with reciprocating syringe instrumentation, continuous flow instrumentation, or high speed mixing equipment. High velocity homogenization or vigorous stirring, producing forces of high shear and cavitation, are preferred. High shear processes are preferred as low shear processes can result in larger API particle sizes. The resultant composition is a composite mixture of API suspended in the emulsion droplet (nanoemulsion fraction) and sterically stabilized micro-/nano-crystalline API in the media. This tri-phasic system comprises particulate drug, oil, and water or buffer. The resultant micro/nano-particulate API has a mean particle size of less than about 3 microns. Smaller particulate API can also be obtained, as described below.

The API can be precipitated out from the oil droplets by adding more of the non-miscible liquid. The precipitated API typically has a mean particle size of less than about 3 microns. If desired, the API particles can be prevented from aggregating or clumping together by incorporating a surfactant or emulsifier, e.g., a “surface stabilizer.”

Route II is utilized for an API that is soluble in at least one part of the emulsion base, such as the solvent. For Route II, an API is dissolved in a mixture of oil, solvent, and stabilizer to form an emulsion pre-mix. The API remains in soluble form if water or buffer is not added to the mixture. Upon the addition of water or buffer and the application of shear forces, the API is precipitated into micro/nano-particles having a mean particle size of less than about 3 microns. Nanoparticles can be produced with reciprocating syringe instrumentation, continuous flow instrumentation, or high speed mixing equipment. High energy input, through high velocity homogenization or vigorous stirring, is a preferred process. The high energy processes reduce the size of the emulsion droplets, thereby exposing a large surface area to the surrounding aqueous environment. High shear processes are preferred, as low shear processes can result in larger particle sizes. This is followed by precipitation of nanoparticulate API previously embedded in the emulsion base. The end product comprises API in solution and particulate suspension, both distributed between the solvent, oil, and water or buffer. Nanoparticulate API has at least one surface stabilizer associated with the surface thereof.

Examples of API that are poorly water soluble in water but soluble in another liquid include estradiol, which is soluble in ethanol, and fenofibrate, which is freely soluble in 1-methyl-2-pyrrolidone or N-methyl-pyrrolidinone [NMP], slightly soluble in oil and stabilizer, and insoluble in water.

If desired, the water miscible oil droplets and API nanoparticles prepared using Route I or Route II can be filtered through either a 0.2 or 0.45 micron filter. Larger oil droplets and/or API particles can be created by simply increasing the water content, decreasing the oil-stabilizer-solvent content, or reducing the shear in forming the oil droplets.

For the emulsion base used in Route I or Route II, the preferred ratio of oil:stabilizer:solvent is about 23:about 5:about 4, respectively, on a weight-to-weight basis. The preferred ratio of the oil comprising phase to water or buffer is about 2: about 1, respectively. According to the present invention, the oil ratio may be about 10 to about 30 parts; the solvent ratio may be about 0.5 to about 10 parts; the stabilizer ratio may be about 1 to about 8 parts, and the water may be about 20 to about 80% (w/w).

B. Definitions

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The phrase “poorly water-soluble drugs” as used herein refers to drugs having a solubility in water of less than about 30 mg/mL, less than about 20 mg/mL, less than about 10 mg/mL, less than about 1 mg/mL, less than about 0.1 mg/mL, less than about 0.01 mg/mL or less than about 0.001 mg/mL.

The phrase “soluble drug” as used herein refers to a drug that has a solubility in water or another media selected from the group consisting of greater than about 10 mg/mL, greater than about 20 mg/mL, and greater than about 30 mg/mL.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

C. Other Preferred Aspects of the Compositions of the Invention

The pharmaceutical compositions of the invention can comprise a macromolecule, e.g., a compound having a molecular weight of greater than about 500 Da. An example of such a compound is cyclosporine. In yet another embodiment of the invention, such pharmaceutical formulations can be delivered to a subject either topically or transdermally.

The pharmaceutical compositions of the invention can comprise an antiviral compound. An example of such an antiviral compound is acyclovir. In another aspect of the invention, the antiviral compositions of the invention can be applied topically or transdermally.

In another embodiment of the invention, the pharmaceutical compositions of the invention have anti-microbial properties. The anti-microbial properties can be associated with the formulation, and not with the active agent. As an example, the formulations described herein, in the absence of an active agent, can exhibit antimicrobial activity.

Antimicrobial agents or preservatives are added to nonsterile dosage forms to protect them from microbiological growth or from microorganisms that are introduced inadvertently during or subsequent to the manufacturing process. In the case of sterile articles used in multi-dose containers, antimicrobial preservatives are added to inhibit the growth of microorganisms that may be introduced by repeatedly withdrawing individual doses.

U.S. Food and Drug Administration guidelines require that antimicrobial effectiveness, whether inherent in the product (e.g., for an antibiotic agent) or whether produced because of the addition of an antimicrobial agent, must be demonstrated for all injections packaged in multiple-dose containers or for other products containing antimicrobial preservatives. Antimicrobial effectiveness must be demonstrated for multiple-dose topical and oral dosage forms, and for other dosage forms such as ophthalmic, otic, nasal, irrigation, and dialysis fluids. See USP 25, Section 51, “Antimicrobial Effectiveness Testing.”

The addition of an antimicrobial agent to a therapeutic dosage form can be undesirable, as such compounds can be toxic and they can have undesirable interactions with the primary active agent to be delivered. In addition, the use of microbicides can promote the generation of drug resistant bacteria, drug resistant yeast, drug resistant fungi, etc. This has been observed with the wide spread use of antibacterial lotions, soaps, cleaning products, etc.

Antibiotic resistance among bacteria has increased in recent years, and concerns have been raised that cross resistance might develop in bacteria or other microorganisms due to exposure to antibiotics or biocides. Rutala, W. A., “APIC Guideline for Selection and Use of Disinfectants,” American J, 24:313-342 (1996); Russell et al., “Do Antiseptics and Disinfectants Select for Antibiotic Resistance?” J. of Medicinal Microbiology, 48:613-615 (1999). More effective disinfectants can be extremely irritant and toxic, resulting in health complications such as contact dermatitis and mucous membrane irritation among personnel. Hansen, K. S., “Occupational Dermatoses in Hospital Cleaning Women,” Contact Dermatitis, 9:343-351 (1983); Beauchamp et al., “A Critical Review of the Toxicology of Glutaraldehyde, Critical Reviews in Toxicology, 22:143-174 (1992). Thus, there is a continuing need for effective and safe biocidal agents for topical and surface disinfection as microorganisms change and resistant strains develop.

The invention is directed to pharmaceutical compositions that surprisingly have antimicrobial, antifungal, antiyeast, and/or antiviral properties. The pharmaceutical compositions of the invention comprise at least one solvent, at least one oil, at least one surface stabilizer (also referred to as a surfactant), and aqueous medium. The compositions additionally may comprise one or more active agents, which may be dissolved or dispersed in any one of the oil, solvent, or water. The active agent can be useful, for example, as a pharmaceutical or cosmetic. No external antibacterial agent or preservative is required to be added to the compositions of the invention to impart the antimicrobial, antiyeast, antifungal, and/or antiviral properties. Moreover, the incorporation of an active agent does not compromise the antimicrobial effectiveness of the compositions of the invention.

The compositions of the invention meet the Antimicrobial Effectiveness Test as described in the United States Pharmacopeia (USP—General chapter # 51). The standard USP testing requires evaluation in five microorganisms: Aspergillus niger (ATCC 16404), Candida albicans (ATCC 10231), Escherichia coli (ATCC 8739), Pseudomonas aeruginosa (ATCC 9027) and Staphylococcus aureus (ATCC 6538).

The compositions of the invention are particularly useful in products used for topical treatment of infections, wound healing, etc., as in addition to the pharmacologic properties of the active agent in such compositions, the vehicle itself acts as a microbicidal agent. Such a composition possibly induces synergistic action and reduces the possibility of development of drug resistance microorganisms. Moreover, such a composition may enable the use of lower doses of the active agent.

At present, various types of cosmetic and pharmaceutical compositions require the addition of an antimicrobial agent to retard microbial growth. Choosing the right antimicrobial agent can be challenging, due to potential interactions between the active agent and the antimicrobial agent. Moreover, the antimicrobial agent can be toxic and it can induce adverse reactions in patients.

D. Exemplary Types of Transdermal Drug Delivery Systems

The invention contemplates several types of transdermal drug delivery systems that are amenable for use in the invention. Any one of the following types of transdermal drug delivery systems, for example, can comprise a suitable API, including the API emulsion formulations disclosed herein, and be placed into contact with the skin of a subject. Over time, depending on the rate of release of the API, the API will be deposited onto or into the skin of the subject from the dosage form, e.g., local delivery, and/or the solubilized form will transport across the skin and into the subject's system, e.g., systemic delivery.

Transdermal drug delivery systems include, but are not limited to, (1) passive drug in adhesive systems, (2) gels, lotions, or creams; (3) thermal systems, which use heat to make the skin more permeable and to increase the energy of the drug molecules, (4) iontophoresis, which uses low voltage electrical current to drive charged drugs through the skin; (5) sonophoresis, which uses low frequency ultrasonic energy to disrupt the stratum corneum; (6) microporation, which includes devices that create micropores in the stratum corneum; (7) electroporation, which uses short electrical pulses of high voltage to crease transient aqueous pores in the skin, or (8) any combination thereof.

A preferred transdermal drug delivery device for the compositions of the invention is a gel, lotion, cream or similar composition to be topically applied. Examples of drugs currently approved or in development for delivery via a gel transdermal drug delivery device include, but are not limited to, alprostadil, dihydrotestosterone, estradiol, and testosterone. One of the challenges for gel/lotion/cream transdermal drug delivery systems is to deliver larger molecules across the skin barrier.

In another embodiment of the invention, the gel/cream/lotion transdermal drug delivery system of the invention can be formulated, or packaged, to provide desired unit dosages, e.g., metered-dose transdermal delivery.

Transdermal patches include, but are not limited to: (1) a single-layer drug-in-adhesive system, (2) a multi-layer drug-in-adhesive system, (3) a reservoir system, and (4) a matrix system. The single-layer drug-in-adhesive system is characterized by the inclusion of the drug directly within the skin-contacting adhesive. In this transdermal system design, the adhesive not only serves to affix the system to the skin, but also serves as the formulation foundation, comprising the drug and all the excipients under a single backing film. The multi-layer drug-in-adhesive is similar to the single-layer drug-in-adhesive in that the drug is incorporated directly into the adhesive. However, the multi-layer encompasses either the addition of a membrane between two distinct drug-in-adhesive layers or the addition of multiple drug-in-adhesive layers under a single backing film. The reservoir transdermal system design is characterized by the inclusion of a liquid compartment comprising a drug solution or suspension separated from the release liner by a semi-permeable membrane and adhesive. The adhesive component of the product responsible for skin adhesion can either be incorporated as a continuous layer between the membrane and the release liner or in a concentric configuration around the membrane. The matrix system design is characterized by the inclusion of a semisolid matrix containing a drug solution or suspension which is in direct contact with the release liner. The component responsible for skin adhesion is incorporated in an overlay and forms a concentric configuration around the semisolid matrix.

E. Compositions of the Invention

The methods of the invention can produce several different types of compositions. A first composition comprises: (1) nanoparticulate API having a mean particle size of less than about 10 microns and having associated with the surface thereof at least one surface stabilizer; (2) water or a buffer; and (3) an emulsion pre-mix or oil phase or lipophilic phase comprising at least one oil and optionally at least one solvent. The composition may additionally comprise microcrystalline API. The particulate API can be present in the water or buffer, oil, solvent, or a combination thereof. Such a composition is made utilizing Route I.

A second composition comprises: (1) nanoparticulate API having a mean particle size of less than about 10 microns and having associated with the surface thereof at least one surface stabilizer; (2) water or buffer; and (3) an emulsion pre-mix or oil phase or lipophilic phase comprising at least one oil, optionally at least one solvent, and solubilized API. The composition may additionally comprise microcrystalline API. The solubilized API can be present in the water or buffer, oil, solvent, or a combination thereof. In addition, nanoparticulate API can be present in the water or buffer, oil, solvent, or a combination thereof. Such a composition is made utilizing Route II. In a further embodiment of the invention, the solubilized API can be precipitated out from the emulsion droplets. The precipitated API has a mean particle size of less than about 10 microns.

The tri-phasic compositions of the invention are beneficial for several reasons. First, formulations resulting from the Route II method comprise both solid and solubilized forms of the same API. This enables a resultant pharmaceutical formulation to provide both immediate release and controlled release of the component API, providing for fast onset of activity combined with prolonged activity of the API. Moreover, when formulated for topical application to the skin, in a cream or lotion for example, the solid API nanoparticles may provide an immediate local therapeutic effect at the skin surface, while the solubilized API within the emulsion base crosses the skin/cell barrier allowing the API to enter the body's system. That is, the solubilized API crosses the skin rapidly and penetrates into deeper layers, whereas the solid part does not permeate into deeper skin layers, but acts as local depot and as a reservoir for supplying drug into deeper layers. Hence, a formulation comprising both API nanoparticles and solubilized API can provide local and systemic therapeutic effects.

The different components of the two types of compositions described above can be separated and used independently.

1. API Nanoparticles

For example, the solid API nanoparticles can be separated from the aqueous suspension media and/or the emulsion globules, for instance, by filtration or centrifugation. This provides a convenient method of obtaining nanoparticles of a poorly water-soluble or water-insoluble API. Furthermore, when a stabilizer is included in the particle size reduction process, it prevents the API nanoparticles from aggregating and, therefore, the API nanoparticles are stabilized at a nanoparticulate size. If desired, the API nanoparticles can then be formulated into any suitable dosage form. API nanoparticles can be made using food grade, USP or NF grade materials suitable for human use applications.

Exemplary dosage forms include, but are not limited to, liquid dispersions, oral suspensions, gels, aerosols, ointments, creams, tablets, capsules, dry powders, multiparticulates, sprinkles, sachets, lozenges, and syrups. Moreover, the dosage forms of the invention include but are not limited to solid dosage forms, liquid dosage forms, semi-liquid dosage forms, immediate release formulations, modified release formulations, controlled release formulations, fast melt formulations, lyophilized formulations, delayed release formulations, extended release formulations, pulsatile release formulations, and mixed immediate release and controlled release formulations, or any combination thereof.

In one embodiment of the invention, the API nanoparticles can be formulated into an aerosol for pulmonary or nasal delivery. The aerosol can be a dry powder aerosol or a liquid dispersion aerosol. The aerosols of the invention can be used for topical, nasal, or pulmonary applications.

In another embodiment of the invention, the therapeutic or diagnostic nanoparticles of the invention can be intravascularly injected into a patient to treat or diagnose local or systemic diseases. The API nanoparticles can also be injected extravascularly to provide controlled release of the nanoparticulate API at the site of injection for prolonged effectiveness, which minimizes the need for multiple dosing.

Since the API nanoparticles have a mean particle size of less than about 3 microns, the particles typically are more readily able to move across absorption barriers, such as mucosal gastrointestinal barriers, nasal, pulmonary, ophthalmic, and vaginal membranes, as compared to microcrystalline API. Similarly, the small API particle size enables passage through blood/tissue and blood/tumor barriers of various organs.

In one embodiment of the invention, the API nanoparticles are fenofibrate nanoparticles.

2. Emulsion Globules Comprising API Nanoparticles and/or Solubilized API

The emulsion globules comprising solubilized API, API nanoparticles, or a combination thereof can also be isolated from the surrounding aqueous or buffer phase and used in therapeutic dosage forms. The emulsion globules can be made using food grade, USP or NF grade materials suitable for human use applications. Nanoparticulate oil globules comprising solubilized API and methods of making the same are described in U.S. Pat. No. 5,629,021 (“the '021 patent”), which is incorporated herein by reference. The emulsion globules of the invention typically comprise (1) solubilized API, particulate API, or a combination thereof; (2) at least one oil; (3) at least one solvent; and (4) at least one surface stabilizer or surfactant. Emulsion globules comprising solubilized API, particulate API, or a combination thereof can be isolated by, for example, filtration. Emulsion globules comprising solubilized API are particularly suitable vehicles for transporting API across the skin barrier and into the blood. Hence, globules comprising solubilized API offer a systemic way to administer API to an individual.

In general, the emulsion globules comprising solubilized API, API nanoparticles, or a combination thereof comprise a significant quantity of API and have diameters of about 10 to about 1000 nm, with a mean a diameter of less than about 1 micron preferred, and with the smallest globules filterable through a 0.2 micron filter, such as is typically used for microbiological purification. The range of API concentration in the globules can be from about 1% to about 50%. The emulsion globules can be stored at between about −20 and about 40° C. In one embodiment of the invention, at least about 50%, at least about 60%, at least about 70%, at least about 80%, or at least about 90% of the globules in the preparation have diameters of less than about 1 micron, less than about 900 nm, less than about 800 nm, less than about 700 nm, less than about 600 nm, less than about 500 nm, less than about 400 nm, less than about 300 nm, less than about 200 nm, or less than about 100 nm.

By varying different parameters of Route I and Route II, the size and integrity of such globules can be modified. Hence, the stability of globules comprising dissolved API can be altered to enable the release of API, either as a solution or precipitate. This is a microreservoir-dissolution-controlled system, where the drug solids act as depot and, as the solubilized fraction is depleted, more drug is drawn into solution form the particulate depot. Thus, the emulsion globules comprising solubilized API enable controlled API release over time.

The small size of the emulsion globules comprising solubilized API, API nanoparticles, or a combination thereof and their compatibility with tissue render them applicable to numerous uses. For example, the emulsion globules are useful as topical drug delivery vehicles as they enable rapid dermal penetration. The globules are also exceptionally versatile in that the API utilized can be any API that is suspendable or dissolvable in any of the water or buffer, oil, or solvent. These properties allow this system to be used with API's that are difficult to formulate for use in other delivery systems.

In addition, the emulsion globules comprising solubilized API, API nanoparticles, or a combination thereof can be diluted with aqueous solutions without stability loss. This enables the use of high API concentration, e.g., up to about 30%, in products which can be diluted for use as necessary. The concentration of API, however, depends on the solubility of the actual drug and the amount of solvent used to dissolve it.

In one embodiment of the invention, the emulsion globules comprise as an API estradiol, acyclovir, or testosterone and are formulated into a dosage form for transdermal delivery.

3. Exemplary Compositions of the Invention

The methods of the invention can produce several different types of compositions to be utilized in the pharmaceutical dosage forms of the invention.

a. Composition 1

A first composition comprises: (1) microparticulate and/or nanoparticulate API particles having a diameter of less than about 10 microns and, optionally for nanoparticulate API, having associated with the surface thereof at least one surface stabilizer; (2) water or a buffer; and (3) an emulsion pre-mix or oil phase or lipophilic phase comprising at least one oil and optionally at least one solvent. The particulate API can be present in the water or buffer, oil, solvent, or a combination thereof. Such a composition is made utilizing Route I.

b. Composition 2

A second composition comprises: (1) microparticulate and/or nanoparticulate API particles having a diameter of less than about 10 microns and, optionally for nanoparticulate API, having associated with the surface thereof at least one surface stabilizer; (2) water or buffer; and (3) an emulsion pre-mix or oil phase or lipophilic phase comprising at least one oil, optionally at least one solvent, and solubilized API. The solubilized API can be present in the water or buffer, oil, solvent, or a combination thereof. In addition, microparticulate and/or nanoparticulate API can be present in the water or buffer, oil, solvent, or a combination thereof. Such a composition is made utilizing Route II. In a further embodiment of the invention, the solubilized API can be precipitated out from the emulsion droplets. The precipitated API has a diameter of less than about 10 microns. In other embodiments of the invention, the precipitated API has a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In other embodiments of the invention, at least about 50%, at least about 60%, at least about 70%, at least about 80%, at least about 90%, at least about 95%, or at least about 99% of the precipitated API can have a diameter less than the size listed above, e.g., less than about 10 microns, less than about 9 microns, etc.

The tri-phasic compositions of the invention are beneficial for several reasons. First, formulations resulting from the Route II method comprise both solid and solubilized forms of the same API. This enables a resultant pharmaceutical formulation to provide both immediate release and controlled release of the component API, providing for fast onset of activity combined with prolonged activity of the API.

Moreover, when formulated for topical application to the skin, in a cream or lotion for example, the solid API microparticles and/or nanoparticles may provide an immediate local therapeutic effect at the skin surface, while the solubilized API within the emulsion base crosses the skin/cell barrier allowing the API to enter the body's system. That is, the solubilized API crosses the skin rapidly and penetrates into deeper layers, whereas the solid API particles do not permeate into deeper skin layers, but act as local depot and as a reservoir for supplying drug into deeper layers. Hence, a formulation comprising both API nanoparticles and/or microparticles, and solubilized API, can provide local and systemic therapeutic effects, which are particularly beneficial for transdermal dosage forms.

The different components of the two types of compositions described above can be separated and, if desired, used independently.

Any suitable API can be formulated into an emulsion-based composition according to the invention. Examples of API include, but are not limited to, acyclovir, cyclosporine, estradiol, cetirizine, nicotine, naltrexone, and alendronic acid, all of which can be utilized in pharmaceutical dosage forms, including but not limited to transdermal drug delivery systems.

4. Exemplary API Nanoparticles and Microparticles

Solid API nanoparticles and microparticles may be separated from the aqueous suspension media and/or the emulsion globules, for instance, by filtration or centrifugation. This provides a convenient method of obtaining nanoparticles and/or microparticles of a poorly water-soluble or water-insoluble API. Furthermore, when a stabilizer is included in the particle size reduction process, it prevents the API nanoparticles from aggregating and, therefore, the API nanoparticles are stabilized at a nanometer size. If desired, the API nanoparticles can then be formulated into any suitable dosage form. API nanoparticles can be made using food grade, USP or NF grade materials suitable for human use applications.

As used herein, API microparticles preferably have a particle size of less than about 10 microns. In other embodiments of the invention, API microparticles have a diameter of less than about 9 microns, less than about 8 microns, less than about 7 microns, less than about 6 microns, less than about 5 microns, less than about 4 microns, less than about 3 microns, less than about 2 microns, or about 1 micron or greater. In other embodiments of the invention, at least about 50%, at least about 60%, at least about 70%, at least about 80%, at least about 90%, at least about 95%, or at least about 99% of the API microparticles can have a diameter less than the size listed above, e.g., less than about 10 microns, less than about 9 microns, etc.

As used herein API nanoparticles have a diameter of less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In other embodiments of the invention, at least about 50%, at least about 60%, at least about 70%, at least about 80%, at least about 90%, at least about 95%, or at least about 99% of the API nanoparticles can have a diameter less than the size listed above, e.g., less than about 1000 nm, less than about 900 nm, etc.

Exemplary dosage forms for pharmaceutical applications, including but not limited to topical or transdermal applications, include, but are not limited to, liquid dispersions, oral suspensions, gels, aerosols, ointments, creams, capsules, dry powders, multiparticulates, sprinkles, sachets, lozenges, and syrups. Moreover, the dosage forms of the invention may be solid dosage forms, liquid dosage forms, semi-liquid dosage forms, immediate release formulations, modified release formulations, controlled release formulations, fast melt formulations, lyophilized formulations, delayed release formulations, extended release formulations, pulsatile release formulations, and mixed immediate release and controlled release formulations, or any combination thereof.

Smaller API microparticles and nanoparticles are preferred, such as those having a diameter of less than about 3 microns, as such particles typically are more readily able to move across absorption barriers, such as skin, as compared to larger API. Similarly, the small API particle size enables passage through blood/tissue and blood/tumor barriers of various organs.

5. Emulsion Globules Comprising API Particles and/or Solubilized API

The emulsion globules comprising solubilized API, API nanoparticles and/or microparticles, or a combination thereof can also be isolated, if desired, from the surrounding aqueous or buffer phase and used in therapeutic dosage forms. The emulsion globules can be made using food grade, USP or NF grade materials suitable for human use applications. Nanoparticulate oil globules comprising solubilized API, and methods of making the same, are described in U.S. Pat. No. 5,629,021 (“the '021 patent”), which is incorporated herein by reference.

The emulsion globules of the invention typically comprise: (1) solubilized API, particulate API, or a combination thereof; (2) at least one oil; (3) at least one solvent; and (4) at least one surface stabilizer or surfactant. Emulsion globules comprising solubilized API, particulate API, or a combination thereof can be isolated, if desired, by, for example, filtration. Emulsion globules comprising solubilized API are particularly suitable vehicles for transporting API across the skin barrier and into the blood. Hence, globules comprising solubilized API offer a systemic way to administer API to an individual.

In general, the emulsion globules comprising solubilized API, API particles, or a combination thereof have a diameter of less than about 10 microns. In other embodiments of the invention, the oil globules can have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In other embodiments of the invention, at least about 50%, at least about 60%, at least about 70%, at least about 80%, at least about 90%, at least about 95%, or at least about 99% of the API microparticles can have a diameter less than the size listed above, e.g., less than about 10 microns, less than about 9 microns, etc.

In a preferred embodiment, the oil globules have a diameter of less than about 2 microns, with a mean diameter of about 1 micron preferred. In another embodiment of the invention, the oil globules are filterable through a 0.2 micron filter, such as is typically used for microbiological purification.

The range of API concentration in the globules can be from about 1% to about 50%. The emulsion globules can be stored at between about −20 and about 40° C.

By varying different parameters of Route I and Route II, the size and integrity of such globules can be modified. Hence, the stability of globules comprising dissolved API can be altered to enable the release of API, either as a solution or precipitate. This is a microreservoir-dissolution-controlled system, where the drug solids acts as depot and, as the solubilized fraction is depleted, more drug is drawn into solution form the particulate depot. Thus, the emulsion globules comprising solubilized API enable controlled API release over time.

The small size of the emulsion globules comprising solubilized API, API nanoparticles and/or microparticles, or a combination thereof and their compatibility with tissue render them applicable to numerous uses. For example, the emulsion globules are useful as topical drug delivery vehicles as they enable rapid dermal penetration. The globules are also exceptionally versatile in that the API utilized can be any API that is suspendable or dissolvable in any of the water or buffer, oil, or solvent. These properties allow this system to be used with API's that are difficult to formulate for use in other delivery systems.

In addition, the emulsion globules comprising solubilized API, API nanoparticles and/or microparticles, or a combination thereof can be diluted with aqueous solutions without stability loss. This enables the use of high API concentration, e.g. up to about 30%, products which can be diluted for use as necessary. The concentration of API, however, depends on the solubility of the actual drug and the amount of solvent used to dissolve it.

In one embodiment of the invention, the emulsion globules comprise as an API estradiol, acyclovir, or testosterone and are formulated into a dosage form for pharmaceutical delivery, including but not limited to transdermal delivery.

F. Methods of Making the Inventive Compositions

Three methods for making the compositions of the invention are described herein. One benefit of the methods of making the compositions to be utilized in the pharmaceutical dosage forms of the invention as compared to prior art methods, such as wet milling, is that the methods are applicable to water-soluble API as well as poorly water-soluble API. Another benefit of the methods of the invention is that they do not require grinding media or specialized grinding process or equipments. The use of such grinding media can add cost and complexity to a particle size reduction process for an API. Additionally, unlike wet milling technologies, the methods of the invention can accommodate amorphous or semi-amorphous API's. In summary, the three methods are as follows: Route I: The API is insoluble or slightly soluble in any of the components of the formulation (e.g., acyclovir); Route II: The API is soluble or partially soluble in at least one of the components of the formulation (e.g., estradiol); and Route III: The API is completely soluble in all of the components of the formulation (e.g., nicotine).

1. Route I

The method of Route I essentially comprises milling an API in an emulsion base. This method requires that the API is poorly soluble or insoluble in all phases of the oil phase/lipophilic phase and the water or buffer. Hence, an API is first suspended in a mixture of a non-miscible liquid, which can comprise at least one oil, at least one solvent, and at least one buffer or water to form an emulsion base, followed by homogenization or vigorous stirring of the emulsion base. API nanoparticles can be produced with reciprocating syringe instrumentation, continuous flow instrumentation, or high speed mixing equipment. High velocity homogenization or vigorous stirring, producing forces of high shear and cavitation, are preferred. High shear processes are preferred as low shear processes can result in larger API particle sizes.

The resultant composition is a composite mixture of API suspended in the emulsion droplet (nanoemulsion API fraction) and sterically stabilized microcrystalline or microparticulate API in the media. This tri-phasic system comprises particulate drug, oil, and water or buffer.

In one embodiment of the invention, the resultant microparticulate API has a diameter of less than about 10 microns, less than about 9 microns, less than about 8 microns, less than about 7 microns, less than about 6 microns, less than about 5 microns, less than about 4 microns, less than about 3 microns, less than about 2 microns, greater than about 1 micron and less than about 2, about 3, about 4, or about 5 microns, or about 1 micron.

In another embodiment of the invention, the nanoparticulate API can have a diameter of less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

In other embodiments of the invention, at least about 50%, at least about 60%, at least about 70%, at least about 80%, at least about 90%, at least about 95%, or at least about 99% of the microcrystalline or microparticulate API in a composition can have a diameter of less than about 10 microns, less than about 9 microns, less than about 8 microns, less than about 7 microns, less than about 6 microns, less than about 5 microns, less than about 4 microns, less than about 3 microns, less than about 2 microns, about 1 micron, or greater than about 1 micron and less than about 2, about 3, about 4, about 5, about 6, about 7, about 8, about 9, or about 10 microns.

In yet other embodiments of the invention, at least about 50%, at least about 60%, at least about 70%, at least about 80%, at least about 90%, at least about 95%, or at least about 99% of the nanoparticulate API can have a diameter of less than about 1000 nm, less than about 900 nm, less than about 800 mm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm in size.

The API can be precipitated out from the oil droplets by adding more of the non-miscible liquid. The precipitated API particles typically have a diameter of less than about 10 microns, less than about 9 microns, less than about 8 microns, less than about 7 microns, less than about 6 microns, less than about 5 microns, less than about 4 microns, less than about 3 microns, less than about 2 microns, or less than about 1 micron. If desired, the API particles can be prevented from aggregating or clumping together by incorporating a surfactant or emulsifier, e.g., a “surface stabilizer.”

2. Route II and Route III

Routes II and III require that the API is soluble or partially soluble in at least one (Route II) or all of the phases (Route III) of the emulsion base; e.g., that the API is soluble in at least one oil, at least one solvent, or water or buffer. In some embodiments, Route II or III can comprise the simultaneous milling and precipitation of an API in an emulsion base.

Route II is utilized for an API that is soluble in at least one part of the emulsion base, such as the solvent, and Route III is utilized for an API that is soluble in all of the components of the emulsion base, such as in water, oil, and a solvent. For Routes II and III, an API is dissolved in a mixture of oil, solvent, and stabilizer to form an emulsion pre-mix. The API remains in soluble form if water or buffer is not added to the mixture. Upon the addition of water or buffer and the application of shear forces, the API is precipitated into microparticles having a diameter of less than about 10 microns, and nanoparticles having a diameter of less than about 1 micron (as described above in Route I; the same particle sizes are applicable to Routes II and III). Nanoparticles can be produced with reciprocating syringe instrumentation, continuous flow instrumentation, or high speed mixing equipment. High energy input, through high velocity homogenization or vigorous stirring, is a preferred process. The high energy processes reduce the size of the emulsion droplets, thereby exposing a large surface area to the surrounding aqueous environment. High shear processes are preferred, as low shear processes can result in larger particle sizes.

This can be followed by precipitation of nanoparticulate API previously embedded in the emulsion base. The end product comprises API in solution and particulate suspension, both distributed between the solvent, oil, and water or buffer. In one embodiment, nanoparticulate API has at least one surface stabilizer associated with the surface thereof.

Examples of API that are poorly water soluble in water but soluble in another liquid include estradiol, which is soluble in ethanol, and fenofibrate, which is freely soluble in 1-methyl-2-pyrrolidone or N-methyl-pyrrolidinone [NMP], slightly soluble in oil and stabilizer, and insoluble in water.

Examples of API that are soluble in all of the components of the compositions of an emulsion base include, e.g., nicotine.

If desired, the water miscible oil droplets and API nanoparticles prepared using Route I, Route II, or Route III can be filtered through either a 0.2 or 0.45 micron filter. Larger oil droplets and/or API particles can be created by simply increasing the water content, decreasing the oil-stabilizer-solvent content, or reducing the shear in forming the oil droplets.

For the emulsion base used in Route I, Route II, or Route III, the preferred ratio of oil:stabilizer:solvent is about 23:about 5:about 4, respectively, on a weight-to-weight basis. The preferred ratio of the oil comprising phase to water or buffer is about 2: about 1, respectively. According to the present invention, the oil ratio may be about 10 to about 30 parts; the solvent ratio may be about 0.5 to about 10 parts; the stabilizer ratio may be about 1 to about 8 parts, and the water may be about 20 to about 80% w/w.

G. Components of the Methods and Compositions of the Invention

1. Active Pharmaceutical Ingredient

a. Properties

Any suitable API may be employed in the compositions and methods of the invention. For an API to be utilized in the Route I method, the API must be poorly soluble or insoluble in all phases of the particle size reduction system, including water and the solvent and oil to be used in the method. For an API to be utilized in Route II, the API must be poorly water-soluble or water insoluble but soluble in at least one phase of the emulsion base, such as the oil or solvent and stabilizer or stabilizer solution. By “poorly water-soluble” or “water insoluble” it is meant that the API has a solubility in water of less than about than about 20 mg/mL, less than about 10 mg/mL, less than about 1 mg/mL, less than about 0.1 mg/mL, less than about 0.01 mg/mL, or less than about 0.001 mg/mL at ambient temperature and pressure and at about pH 7.

The API to be used in the methods of the invention, and present in the compositions of the invention, can be amorphous, semi-amorphous, crystalline, semi-crystalline, or a mixture thereof.

b. API Particle Size

As used herein, API particle size is determined on the basis of the weight average particle size as measured by conventional techniques well known to those skilled in the art, such as sedimentation field flow fractionation, laser diffraction, photon correlation spectroscopy (also known as dynamic light scattering), electroacoustic spectroscopy, or disk centrifugation.

As used herein, “nanoparticulate API” refers to API having a diameter of less than about 1 micron. “Microcrystalline API” refers to API having a diameter of greater than about 1 micron. In other embodiments of the invention, microparticulate API have a diameter of less than about 10 microns, less than about 9 microns, less than about 8 microns, less than about 7 microns, less than about 6 microns, less than about 5 microns, less than about 4 microns, less than about 3 microns, less than about 2 microns, or about 1 micron or greater. In other embodiments of the invention, at least about 50%, at least about 60%, at least about 70%, at least about 80%, at least about 90%, at least about 95%, or at least about 99% of the API microparticles can have a diameter less than the size listed above, e.g., less than about 10 microns, less than about 9 microns, etc.

In yet other embodiments of the invention, nanoparticulate API has a diameter of less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 mm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In other embodiments of the invention, at least about 50%, at least about 60%, at least about 70%, at least about 80%, at least about 90%, at least about 95%, or at least about 99% of the API nanoparticles can have a diameter less than the size listed above, e.g., less than about 900 nm, less than about 800 nm, etc.

In other embodiments of the invention, at least about 60%, at least about 70%, at least about 80%, at least about 90%, at least about 95%, or at least about 99% of the API particles, or droplets comprising solubilized API, have a size less than the mean particle size, less than about 3 microns, less than about 2900 nm, less than about 2800 nm, etc.

c. Exemplary API

Any suitable API may be used in the methods and compositions of the invention. Examples of classes of useful API include, but are not limited to, therapeutic and diagnostic agents, pigments, paints, inks, dyes, photographic materials, cosmetic ingredients, etc.

In one embodiment of the invention, the API is estradiol, fenofibrate, acyclovir, alendronic acid, or testosterone. Specific examples of APIs that may be utilized in the methods of the invention include, but are not limited to, insulin, calcitonin, calcitonin gene regulating protein, atrial natriuretic protein, betaserori, erythropoietin, alpha interferon, beta interferon, gamma interferon, somatropin, somatotropin, somastostatin, insulin-like growth factor, luteinizing hormone releasing hormone, factor VIII, interleukins, interleukin analogues, hematological agents, anticoagulants, hematopoietic agents, hemostatics, thrombolytic agents, endocrine agents, antidiabetic agents, antithyroid agents, beta-adrenoceptor blocking agents, growth hormones, growth hormone releasing hormone, sex hormones, thyroid agents, parathyroid calcitonin, biphosphonates, uterine-active agents, cardiovascular agents, antiarrhythmic agents, anti-anginal agents, anti-hypertensive agents, vasodilators, agents used in treatment of heart disorders, cardiac inotropic agents, renal agents, genitounnary agents, antidiuretic agents, respiratory agents, antihistamines, cough suppressants, parasympathomimetics, sympathomimetics, xanthines, central nervous system agents, analgesics, anesthetics, anti-emetic agents, anorexiants, antidepressants, anti-migraine agents, antiepileptics, dopaminergics, anticholinergics, antiparkinsonian agents, muscle relaxants, narcotic antagonists, sedatives, stimulants, treatments for attention deficit disorder, methylphenidate, fluoxamine, bisolperol, tacrolimus, sacrolimus, cyclosporine, gastrointestinal agents, systemic anti-infectives, agents used in the treatment of AIDS, anthelmintics, antimycobacterial agents, immunologic agents, vaccines, hormones; dermatological agents including, anti-inflammatory agents, elastase inhibitors, antimuscarinic agents, lipid regulating agents, blood products, blood substitutes, antineoplastic agents including, leuprolide acetate, chemotherapy agents, oncology therapies, nutrients, nutritional agents, chelating agents, interleukin-2, IL-1ra, heparin, hirudin, colony stimulating factors, tissue plasminogen activator, estradiol, oxytocin, nitroglycerine, diltiazem, clonidine, nifedipine, verapamil, isosorbide-5-mononitrate, organic nitrates, diuretics, desmopressin, vasopressin, expectorants, mucolytics, fentanyl, sufentanil, butorphanol, buprenorphine, levorphanol, morphine, hydromorphone, hydrocodone, oxymorphone, methadone, lidocaine, bupivacaine, diclofenac, naproxen, paverin, scopolamine, ondansetron, domperidone, metoclopramide, sumatriptan, ergot alkaloids, benzodiazepines, phenothiozines, prostaglandins antibiotics, antiviral agents, anti-fungals, immunosuppressants, anti-allergic agents, e.g., loratadine and desloratadine, astringents, corticosteroids fluorouracil, bleomycin, vincristine, or deferoxamine.

The API may be a hormone, such as testosterone, progesterone, and estrogen. Other hormones include: (1) Amine-derived hormones, such as catecholamines, adrenaline (or epinephrine), dopamine, noradrenaline (or norepinephrine), tryptophan derivatives, melatonin (N-acetyl-5-methoxytryptamine), serotonin (5-HT), tyrosine derivatives, thyroxine (T4), triiodothyronine (T3); (2) peptide hormones, such as antimullerian hormone (AMH, also mullerian inhibiting factor or hormone), adiponectin (also Acrp30), adrenocorticotropic hormone (ACTH, also corticotropin), angiotensinogen and angiotensin, antidiuretic hormone (ADH, also vasopressin, arginine vasopressin, AVP), atrial-natriuretic peptide (ANP, also atriopeptin), Calcitonin, cholecystokinin (CCK), corticotropin-releasing hormone (CRH), erythropoietin (EPO), follicle-stimulating hormone (FSH), gastrin, glucagons, gonadotropin-releasing hormone (GnRH), growth hormone-releasing hormone (GHRH), human chorionic gonadotropin (hCG), growth hormone (GH or hGH), insulin, insulin-like growth factor (IGF, also somatomedin), leptin, luteinizing hormone (LH), melanocyte stimulating hormone (MSH or α-MSH), neuropeptide Y, oxytocin, parathyroid hormone (PTH), prolactin (PRL), relaxin, rennin, secretin, somatostatin, thrombopoietin, thyroid-stimulating hormone (TSH), thyrotropin-releasing hormone (TRH); (3) steroid hormones, such as glucocorticoids, cortisol, mineralocorticoids, aldosterone, sex steroids, androgens, testosterone, dehydroepiandrosterone (DHEA), dehydroepiandrosterone sulfate (DHEAS), androstenedione, dihydrotestosterone (DHT), Estrogens, estradiol, Progestagens, progesterone, Progestins, (4) sterol hormones, such as vitamin D derivatives and calcitriol, (5) lipid and phospholipid hormones (eicosanoids) such as prostaglandins, leukotrienes, prostacyclin, and thromboxane.

Since the first transdermal patch was approved in 1981 to prevent the nausea and vomiting associated with motion sickness, the U.S. Food and Drug Administration (FDA) has approved, throughout the past 22 years, more than 35 transdermal patch products, spanning numerous molecules, such as fentanyl, nitroglycerin, estradiol, ethinyl estradiol, norethindrone acetate, testosterone, clonidine, nicotine, lidocaine, prilocalne, scopolamine, norelgestromin, and oxybutynin. See Gordon et al., “4 Myths About Transdermal Drug Delivery,” Transdermal Delivery (www.drugdeliverytech.com). Table 1 below provides examples of compounds being developed for transdermal delivery. The drugs described below can be utilized in the compositions and methods of the invention.

TABLE 1
Transdermal products that are in clinical development in the United States.

| Compound | TDD Technology | Development State |
|---|---|---|
| alprostadil | Gel | Preclinical |
| buprenorphine | Patch | Phase III |
| dexamethasone | Iontophoresis | Phase III |
| dextroamphetamine | Patch | Preclinical |
| diclofenac | Patch | Preclinical |
| dihydrotestosterone | Gel | Phase III |
| estradiol | Gel | Phase III |
| androgen/estradiol | Patch | Phase II |
| estradiol/progestin | Patch | Submitted NDA |
| testosterone/estradiol | Patch | Phase III |
| fentanyl | Patch, | Preclinical to |
| | Iontophoresis | Phase III |
| flurbiprofen | Patch | Preclinical |
| lidocaine | Iontophoresis | Phase III |
| glucagon-like peptide-I | Microneedle | Preclinical |
| methylphenidate | Patch | Submitted NDA |
| parathyroid hormone | Microneedle | Preclinical |
| rotigotine | Patch | Phase III |
| testosterone | Gel | Preclinical to |
| | | Submitted NDA |
| Unknown compound for | Patch | Phase III |
| treatment of onychomycosis | | |
| Vaccines (various) | Patch | Preclinical |
| Various (macromolecules, etc.) | Sonophoresis | Preclinical |

Amphiphile-type APIs may be incorporated into the present formulations. That is, drugs or therapeutic compounds that can be ionized and are soluble in polar or non-polar solvents may be incorporated in the formulations of the present invention. Such compounds are soluble, therefore, both in oil and aqueous environments (amphiphiles). Examples of such compounds include nicotine and cetirizine.

Hydrophilic APIs also may be incorporated into a formulation of the present invention. Such compounds include, but are not limited to naltrexone hydrochloride, alendronic acid, and cetirizine dihydrochloride.

2. Oils

For both the methods of Route I and Route II and the compositions of the invention, any suitable oil can be used. Exemplary oils that can be used include, for example, vegetable oils, nut oils, fish oils, lard oil, mineral oils, squalane, tricaprylin, and mixtures thereof. Specific examples of oils that may be used include, but are not limited to, almond oil (sweet), apricot seed oil, borage oil, canola oil, coconut oil, corn oil, cotton seed oil, fish oil, jojoba bean oil, lard oil, linseed oil (boiled), Macadamia nut oil, medium chain triglycerides, mineral oil, olive oil, peanut oil, safflower oil, sesame oil, soybean oil, squalene, sunflower seed oil, tricaprylin (1,2,3-trioctanoyl glycerol), wheat germ oil, and mixtures thereof.

3. Stabilizers or Surfactants

The stabilizer used in the methods and compositions of the invention associates with, or adsorbs, to the surface of the nanoparticulate API, but does not covalently bind to the API. In addition, the individual stabilizer molecules are preferably free of cross-linkages. The stabilizer is preferably soluble in water. One or more stabilizers may be used in the compositions and methods of the invention. As used herein, the terms “stabilizer”, “surface stabilizer”, and “surfactant” are used interchangeably.

Any suitable nonionic or ionic surfactant may be utilized in the compositions of the invention, including anionic, cationic, and zwitterionic surfactants. Exemplary stabilizers or surfactants that may be used in both Routes I and II include, but are not limited to, non-phospholipid surfactants, such as the Tween (polyoxyethylene derivatives of sorbitan fatty acid esters) family of surfactants (e.g., Tween 20, Tween 60, and Tween 80), nonphenol polyethylene glycol ethers, sorbitan esters (such as Span and Arlacel), glycerol esters (such as glycerin monostearate), polyethylene glycol esters (such as polyethylene glycol stearate), block polymers (such as Pluronics), acrylic polymers (such as Pemulen), ethoxylated fatty esters (such as Cremophor RH-40), ethoxylated alcohols (such as Brij), ethoxylated fatty acids, monoglycerides, silicon based surfactants, polysorbates, Tergitol NP-40 (Poly(oxy-1,2-ethanediyl), α-(4-nonylphenol)-.omega.-hydroxy, branched [molecular weight average [980]), and Tergitol NP-70 (a mixed surfactant—AQ=70%).

4. Solvents

Any suitable solvent can be used in the methods and compositions of the invention. Exemplary solvents include, but are not limited, to isopropyl myristate, triacetin, N-methyl pyrrolidinone, aliphatic or aromatic alcohols, polyethylene glycols, propylene glycol. An example of an alcohol useful in the present invention includes, but is not limited to ethanol. Other short chain alcohols and/or amides may be used. Other solvents include dimethyl sulfoxide, dimethyl acetamide, and ethoxydiglycol. Mixtures of solvents can also be used in the compositions and methods of the invention.

5. Water or Buffer

If the methods and/or compositions of the invention use or comprise water or a buffer, the aqueous solution is preferably a physiologically compatible solution such as water or phosphate buffered saline.

6. Other Ingredients

A number of other materials may be added to the compositions of the invention. Volatile oils, such as volatile flavor oils, can be used in lieu of some of the oil or can be added in addition to the primary oil. Exemplary volatile oils or fragrances that can be utilized in the invention include, but are not limited to, balm oil, bay oil, bergamot oil, cedarwood oil, cherry oil, cinnamon oil, clove oil, origanum oil, and peppermint oil. A coloring agent, such as a food coloring agent can also be used. Exemplary food colors that can be utilized in the compositions of the invention include, but are not limited to, green, yellow, red, and blue. The food colors utilized are food grade materials (McCormick), although materials from other sources can be substituted. In addition, a flavoring extract can be used in the methods and compositions of the invention. Exemplary flavored extracts include, but are not limited to, pure anise extract (73% alcohol), imitation banana extract (40% ethanol), imitation cherry extract (24% ethanol), chocolate extract (23% ethanol), pure lemon extract (84% ethanol), pure orange extract (80% ethanol), pure peppermint extract (89% ethanol), imitation pineapple extract (42% ethanol), imitation rum extract (35% ethanol), imitation strawberry extract (30% ethanol), and pure or imitation vanilla extract (35% ethanol). Typically, the extracts utilized are food grade materials (McCormick), although materials from other sources can be substituted.

H. Methods of Using the Compositions of the Invention

The compositions of the invention can be administered to a subject via any conventional means including, but not limited to, orally, rectally, ocularly, optically, e.g., via the eye, parenterally (e.g., intravenous, intramuscular, or subcutaneous), intranasal, colonic, intracisternally, pulmonary, vaginally, intraperitoneally, transdermally, locally (e.g., powders, creams, ointments or drops), topically, or as a buccal or nasal spray. As used herein, the term “subject” is used to mean an animal, preferably a mammal, including a human or non-human. The terms patient and subject may be used interchangeably. In addition, the compositions of the invention can be formulated into any suitable dosage form, such as liquid dispersions, oral suspensions, gels, aerosols, ointments, creams, tablets, capsules, dry powders, multiparticulates, sprinkles, sachets, lozenges, and syrups. Moreover, the dosage forms of the invention may be solid dosage forms, liquid dosage forms, semi-liquid dosage forms, immediate release formulations, modified release formulations, controlled release formulations, fast melt formulations, lyophilized formulations, delayed release formulations, extended release formulations, pulsatile release formulations, and mixed immediate release and controlled release formulations, or any combination thereof.

Of particular importance is the ability to transmit drugs topically or transdermally. It has been known for many years that small particles, such as those below one micron in diameter, can more easily traverse the skin boundary than larger particles. However, the small amount of drug transmitted in small particles has often limited their usefulness. In addition, most particles have only had limited classes of materials they could deliver.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The compositions of the invention may also comprise adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include tonicity agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the compositions of the invention may be admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the API, the liquid dosage forms may comprise inert diluents commonly used in the art, such as water or other solvents, solubilizing agents, and emulsifiers. Exemplary emulsifiers and solvents include, but are not limited to ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like. Besides such inert diluents, the composition can also include adjuvants, such as wetting agents, emulsifying and suspending agents, sweetening, flavoring, and perfuming agents.

“Therapeutically effective amount” as used herein with respect to an API dosage shall mean that dosage that provides the specific pharmacological response for which the API is administered in a significant number of subjects in need of such treatment. It is emphasized that “therapeutically effective amount,” administered to a particular subject in a particular instance may not be effective for 100% of patients treated for a specific disease, and will not always be effective in treating the diseases described herein, even though such dosage is deemed a “therapeutically effective amount” by those skilled in the art.

One of ordinary skill will appreciate that effective amounts of an API can be determined empirically and can be employed in pure form or, where such forms exist, in pharmaceutically acceptable salt, ester, or prodrug form. Actual dosage levels of an API in the nanoparticulate compositions of the invention may be varied to obtain an amount of the API that is effective to obtain a desired therapeutic response for a particular composition and method of administration. The selected dosage level therefore depends upon the desired therapeutic effect, the route of administration, the potency of the administered API, the desired duration of treatment, and other factors.

Dosage unit compositions may comprise such amounts or submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

I. Modulation of Active Pharmaceutical Ingredient Release

It is possible to vary the components of the compositions described herein to modulate the rate at which a particular API is released from the formulation. Thus, it is possible to prepare compositions having different rates of API flux. This enables preparation of a transdermal device or composition, for example, or a topical semisolid cream that is suited to a particularly desired rate of transdermal drug administration.

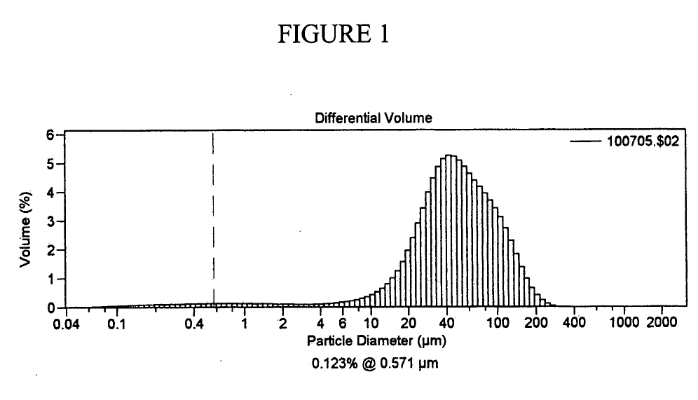

Factors such as the type of oil utilized, the type of solvent utilized, and presence of API solid crystals in a formulation can change the rate of API flux from the transdermal device, cream or semisolid into the skin. Other factors include emulsion droplet size, pH, salt form, and ratio of solid API particles to solubilized API. See, for instance, Example 2 and FIG. 1 which illustrates the results of in vitro studies of ethanol versus N-methyl pyrrolidinone on the rate of release of estradiol.

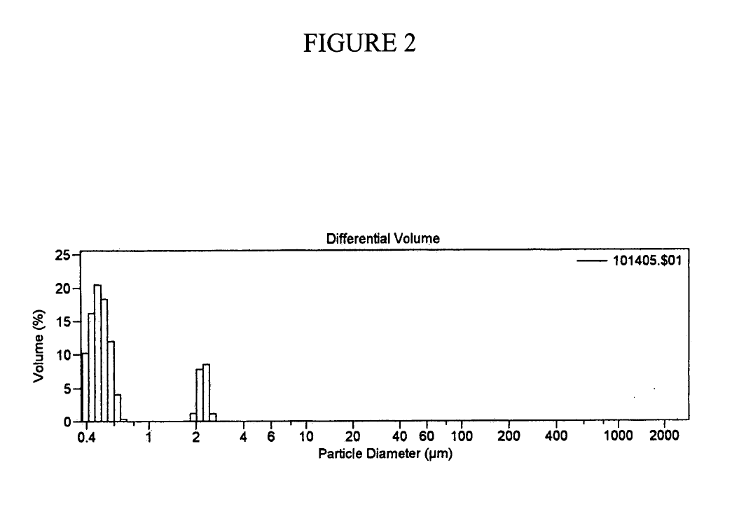

FIG. 2 similarly depicts the effect of the presence of solid estradiol crystals in the formulation on the rate of estradiol release. In the composition that does not comprise crystalline estradiol, the rate of release of estradiol is slower and less hormone is released per microgram of square centimeter of cadaver skin.

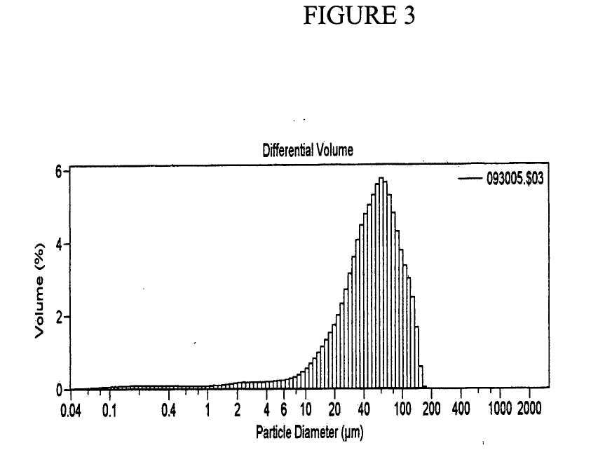

Likewise, FIG. 3 depicts the effect of oil on a variety of estradiol formulations. Accordingly, it is possible to use different types of such formulation components, in different amounts, or different phases (e.g., solubilized versus solid) to change the rate and amount of which any desired active pharmaceutical ingredient is released from the formulation and deposited onto or through the skin.

J. Thermostable Micellar Nanoparticle Compositions

In one aspect of the invention, the compositions of the invention are stable following exposure to elevated temperatures (e.g., “thermostable” compositions). Such compositions can be utilized in any desired pharmaceutical dosage form. Exemplary pharmaceutical dosage forms include, but are not limited to: (1) dosage forms for administration selected from the group consisting of oral, pulmonary, intravenous, rectal, otic, opthalmic, colonic, parenteral, intracisternal, intravaginal, intraperitoneal, local, buccal, nasal, and topical administration; (2) dosage forms selected from the group consisting of liquid dispersions, gels, aerosols, ointments, creams, tablets, sachets and capsules; (3) dosage forms selected from the group consisting of lyophilized formulations, fast melt formulations, controlled release formulations, delayed release formulations, extended release formulations, pulsatile release formulations, and mixed immediate release and controlled release formulations; or (4) any combination thereof. In one embodiment, the dosage form is a transdermal dosage form.

“Thermostable” compositions can be stable against chemical instability, physical instability, or a combination thereof. “Physical” instability refers to phase separation and/or particle agglomeration following exposure to elevated temperatures. “Chemical” stability refers to the chemical stability of a compound following exposure to elevated temperatures; e.g., the composition does not oxidize or otherwise chemically change. Thus, in one embodiment of the invention the thermostable compositions retain their structural integrity following exposure to elevated temperatures. In another embodiment, the thermostable compositions retain their chemical integrity following exposure to elevated temperatures, and in a third embodiment the thermostable compositions retain their structural and chemical integrity following exposure to elevated temperatures.

In one embodiment of the invention, the elevated temperature is sufficient in temperature and duration to sterilize the composition, e.g., conventional autoclaving at 121° C. Two accepted methods (there are others, e.g., gamma irradiation) for sterilizing pharmaceutical products are heat sterilization and sterile filtration. Sterile filtration is an effective method for sterilizing solutions having a particle size of less than 0.2 microns (200 nm), because a 0.2 micron mesh size filter is sufficient to remove most bacteria. However, many desirable compositions may have an effective average particle size of greater than 200 nm. and/or due to their shape, cannot be effectively sterilized by conventional filters.

Sterile filtration is less desirable than conventional autoclaving (steam heat) at 121° C. This is because with heat sterilization, the pharmaceutical composition can be placed in the final storage container and sterilized (a single-step process). The product can then be marketed in the heat sterilized container. In contrast, the filter-sterilization step of sterile filtration is followed by a packaging step (a two-step process). The secondary packaging step of sterile filtration substantially increases the risk of contamination as compared to conventional autoclaving. For these reasons, the Food and Drug Administration generally requires submission of data demonstrating that a formulation cannot be autoclaved before approval of sterile filtration as a method of sterilization for a sterile product.

Micellar nanoparticles are quite viscous and cannot be readily sterilized using aseptic filtration devices, such as filtration using a 0.2 micron filter. Terminal heat sterilization, however, is a desirable method for sterilizing such pharmaceutical compositions. A problem is that, typically, micellar nanoparticle formulations are not stable at elevated temperatures, e.g., at temperatures above 50° C., and therefore cannot be readily autoclaved. The present invention however provides micellar nanoparticle drug compositions which are heat stabile and therefore amenable to heat sterilization.

The compositions of the invention can be stable when exposed to a temperature selected from the group consisting of greater than about 50° C., greater than about 55° C., greater than about 60° C., greater than about 65° C., greater than about 70° C., greater than about 75° C., greater than about 80° C., greater than about 85° C., greater than about 90° C., greater than about 95° C., greater than about 100° C., greater than about 105° C., greater than about 110° C., greater than about 115° C., greater than about 120° C., greater than about 125° C., greater than about 130° C., greater than about 135° C., greater than about 140° C., greater than about 145° C., or greater than about 150° C.

In addition, the compositions of the invention can be stable when exposed to an elevated temperature for a duration of time selected from the group consisting of about 1 minute or less, about 2 minutes or less, about 3 minutes or less, about 4 minutes or less, about 5 minutes or less, about 6 minutes or less, about 7 minutes or less, about 8 minutes or less, about 9 minutes or less, about 10 minutes or less, about 11 minutes or less, about 12 minutes or less, about 13 minutes or less, about 14 minutes or less, about 15 minutes or less, about 16 minutes or less, about 17 minutes or less, about 18 minutes or less, about 19 minutes or less, about 20 minutes or less, about 25 minutes or less, about 30 minutes or less, about 35 minutes or less, about 40 minutes or less, about 45 minutes or less, about 50 minutes or less, about 55 minutes or less, about 60 minutes or less.

Exemplary thermostable surfactants and/or stabilizers include, but are not limited to, (1) sorbitan esters, such as Spans and Arlacel, (2) block polymers, such as Pluronics, (3) acrylic polymers, such as Pemulen, and (4) ethoxylated fatty esters, such as Cremophor RH-40.

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### EXAMPLE 1

The purpose of this example was to prepare a nanoparticulate fenofibrate composition using the Route II method of the invention. Fenofibrate is insoluble in water. The compound produces reductions in total cholesterol, LDL cholesterol, apo-lipoprotein B, total triglycerides, and triglyceride rich lipoprotein (VLDL) in treated patients. In addition, treatment with fenofibrate results in increases in high density lipoprotein (HDL) and apolipoprotein apoAI and apoAII. See The Physicians' Desk Reference, 56^{th} Ed., pp. 513-516 (2002).

The raw fenofibrate particles had a mean particle size of 57 μm, as shown in the particle size distribution of raw fenofibrate given in FIG. 1. A Coulter particle sizer LS230 was used to measure particle size.

4.8 g of fenofibrate were dissolved in 7.0 g of N-methyl-pyrrolidinone (NMP). 41.8 g of medium chain triglycerides (Crodamol GTCC, Croda) were then added to the fenofibrate solution. 9.5 g of Pluronic® F-68, which is a surfactant, was dissolved in 37.0 g of water, and the surfactant solution was then added to the fenofibrate solution. The resultant mixture was then mixed well using a mechanical stirrer for about 15 minutes. The mixture was then fed into a high-pressure homogenizer (APV Invensys, model APV-1000), and the pressure was tuned to 10,000 psi. The mixture was run through the homogenizer for three passes.

As shown in FIG. 2, the resultant mean particle size of nanoemulsion droplets comprising fenofibrate was 60 nm, with 100% of the fenofibrate particles having a size of less than 3 microns.

### EXAMPLE 2

The purpose of this example was to prepare a nanoparticulate estradiol composition using the Route II method of the invention. Estradiol (17b-estradiol) is a white, crystalline, solid, chemically described as estra-1,3,5(10)-triene-3,17b-diol. The compound is poorly water-soluble. Estradiol is indicated for use in Hormone Replacement Therapy, as well as in treating transsexuals.

0.25 g of estradiol were dissolved in 8.8 g of ethanol. The mean particle size for raw estradiol is about 542 microns. 50.2 g of soybean oil and 9.4 g of polysorbate 80, which is a surfactant, were then added to the estradiol solution. The resultant mixture was then mixed well using a mechanical stirrer for about 15 minutes. The mixture was then fed into a high-pressure homogenizer (APV Invensys, model APV-1000), and the pressure was tuned to 10,000 psi. The mixture was run through the homogenizer for two passes.

The resulting emulsion composition exhibited a mean estradiol-comprising droplet size of about 93 nm.

### EXAMPLE 3

The purpose of this example was to prepare a nanoparticulate alendronic acid composition using the Route I method of the invention. Alendronic acid is a bisphosphonate used to treat osteoporosis. It is a white crystalline powder which is insoluble in water. The mean particle size of raw alendronic acid is about 190-210 μm.

1.0 g of alendronic acid was mixed with 8.8 g of ethanol, 9.4 g of polysorbate 80, and 50.2 g of soybean oil. 30.6 g of water was then added to the alendronic acid mixture. Alendronic acid is not soluble in ethanol. The resultant mixture was then mixed well using a mechanical stirrer for about 15 minutes. The mixture was then fed into a high-pressure homogenizer (APV Invensys, model APV-1000), and the pressure was tuned to 10,000 psi. The mixture was run through the homogenizer for two passes.

The resulting milled mean particle size of the alendronic acid was about 0.2 μm.

### EXAMPLE 4

The purpose of this example was to prepare a nanoparticulate acyclovir composition using the Route II method of the invention. Acyclovir is an antiviral used to treat herpes infections of the skin, lip, and genitals; herpes zoster (shingles); and chickenpox. The drug is formulated as oral and topical dosage forms. Acyclovir is moderately soluble in water.

The raw acyclovir particles had a mean particle size of 54 μm, as shown in the particle size distribution of raw acyclovir given in FIG. 3.

5.0 g of acyclovir was partially dissolved in 10.0 g N-methyl-pyrrolidinone (NMP). 47.5 g of mineral oil (light) and 9.4 g of polysorbate 80 were then added to the acyclovir mixture. 27.9 g of water was then added, and the resultant mixture was mixed well using a mechanical stirrer for about 15 minutes. The mixture was then fed into a high-pressure homogenizer (APV Invensys, model APV-1000), and the pressure was tuned to 10,000 psi. The mixture was run through the homogenizer for two passes.

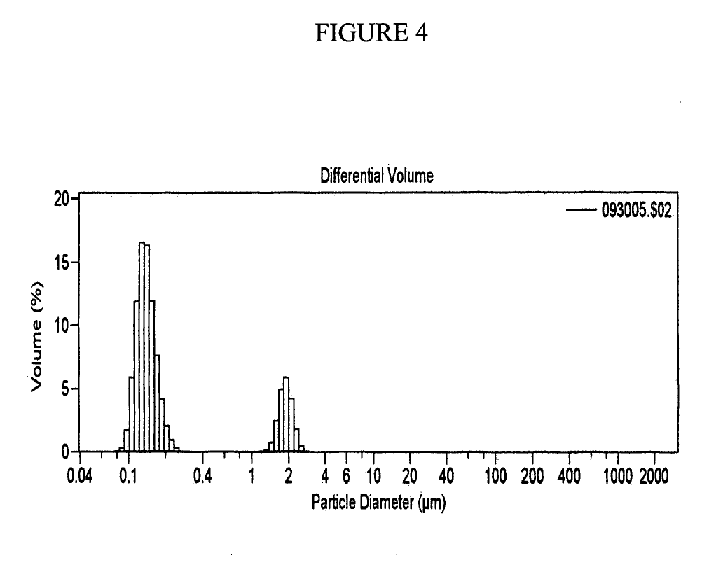

As shown in FIG. 4, the resultant mean particle size of acyclovir within the emulsion droplets was 132 nm, with 100% of the acyclovir particles having a mean size of less than 3 microns.

### EXAMPLE 5

The purpose of this example was to prepare a nanoparticulate fenofibrate composition using the Route II method of the invention. As noted above, the raw fenofibrate particles had a mean particle size of 54 μm. See FIG. 1.

4.8 g of fenofibrate was partially dissolved in 8.8 g of ethanol. 50.2 g of soybean oil and 9.4 g of polysorbate 80 were then added to the fenofibrate mixture. Next, 26.8 g of water was added, and the mixture was mixed well using a mechanical stirrer for about 15 minutes. The mixture was then fed into a high-pressure homogenizer (APV Invensys, model APV-1000), and the pressure was tuned to 10,000 psi. The mixture was run through the homogenizer for three passes. The milled fenofibrate had a mean particle size of about 2 microns.

### EXAMPLE 6

The purpose of this example was to prepare a nanoparticulate acyclovir composition using the Route I method of the invention. As noted above, the raw acyclovir particles had a mean particle size of 54 μm. See FIG. 3.

5.0 g of acyclovir was mixed with 8.8 g of ethanol, 47.7 g of soybean oil, and 9.4 g of polysorbate 80. 29.2 g of water was then added to the acyclovir mixture. The resultant mixture was mixed well using a mechanical stirrer for about 15 minutes. The mixture was then fed into a high-pressure homogenizer (APV Invensys, model APV-1000), and the pressure was tuned to 10,000 psi. The mixture was run through the homogenizer for two passes.

The resultant mean particle size of the milled acyclovir was about 2 microns.

### EXAMPLE 7

The purpose of this example was to prepare a nanoparticulate raloxifene composition using the Route II method of the invention. Raloxifene is a selective estrogen receptor modulator (SERM) that belongs to the benzothiophene class of compounds. The drug is used in the treatment and prevention of postmenopausal osteoporosis. Raloxifene is very slightly soluble in water. The mean particle size for raw raloxifene is about 15-30 μm.

1.0 g of raloxifene was partially dissolved in 20.0 g of ethanol. 40.0 g of mineral oil and 9.4 g of polysorbate 80 were added to the raloxifene mixture. 29.6 g of water was then added to the raloxifene mixture. The resultant mixture was mixed well using a mechanical stirrer for about 15 minutes. The mixture was then fed into a high-pressure homogenizer (APV Invensys, model APV-1000), and the pressure was tuned to 10,000 psi. The mixture was run through the homogenizer for two passes. The mean particle size of the milled raloxifene is 100% (by volume) below 10 microns and mean (by volume) is 2.79 microns.

### EXAMPLE 8

The purpose of this example was to evaluate the transdermal delivery of the acyclovir compositions as prepared in Examples 4 and 6 above as compared to a commercial, non-nanoparticulate form of acyclovir, ZOVIRAX®. ZOVIRAX® is a topical cream formulation.

50 mg of the three different formulations (Composition 4, Composition 6, and ZOVIRAX®) were applied onto cadaver skin on Franz diffusion cells. The exposed surface area was 1.77 sq·cm, and the drug in receptor compartment was measured using HPLC against time.

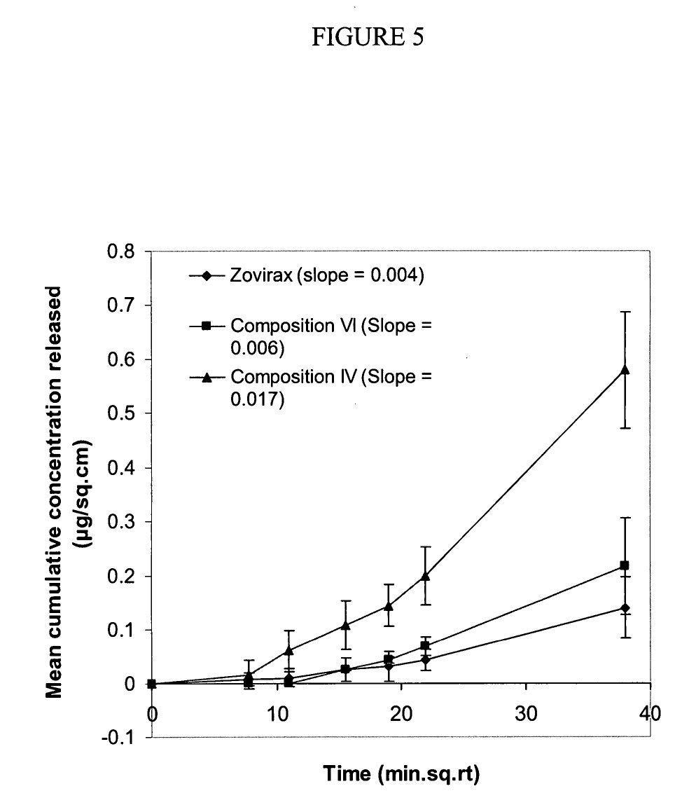

The mean cumulative concentration of acyclovir released over time is graphically shown in FIG. 5. The slope of the release rate for Compositions 4, 6 and ZOVIRAX® was 0.017, 0.006, and 0.004, respectively. The findings indicate a higher retention of drug in the epidermal layer of the skin as well as a higher flux of the drug across that skin barrier and into the body for Compositions 4 and 6 as compared to ZOVIRAX®. Thus, the compositions of the invention exhibited superior in vitro drug disposition profile for acyclovir as compared to the non-nanoparticulate convention acyclovir formulation.

### EXAMPLE 9

The purpose of this example was to evaluate the effectiveness of oral delivery of a fenofibrate formulation prepared according to the invention as compared to a commercial formulation of nanoparticulate fenofibrate, TRICOR® (Abbott Laboratories).

Two fenofibrate formulations according to the invention were tested: the formulations prepared in example # 4 (composition II) and example # 1 (Composition I).

For a control formulation, an oral liquid formulation comprised fenofibrate suspended in a 0.5% (w/w) solution of hydroxypropylmethyl cellulose (HPMC). A 0.5% (w/w) HPMC E4M solution was used as a vehicle to administer fenofibrate. The particle size was same as the raw fenofibrate.

Four Groups of subjects were tested, with five rats per group. Group I received Composition I, Group 2 received Composition II, Group 3 received the standard TRICOR® formulation, and Group 4 received the control formulation. The control group was fed with HPMC gel containing fenofibrate. Each rat was given a single dose of 90 mg/kg of fenofibrate under fasting conditions. The AUC over a 24 period (correlating to the amount of drug absorbed or bioavailability), Cₘₐₓ (maximum concentration of the drug in the blood), Tₘₐₓ (time to reach Cₘₐₓ), T_{1/2} (oral) and CL/F (drug clearance expressed as a function of bioavailability) were measured for each of the four Groups, as shown in the table below. T_{1/2} refers to the elimination half-life.

TABLE 2

| | | | | | | | T_{1/2} | | | |
|---|---|---|---|---|---|---|---|---|---|---|
| | AUC₂₄ₕ | | Cₘₐₓ | | Tₘₐₓ | | Oral | | CL/F | |
| Group | (hr · ng/mL) | | (ng/mL) | | (hr) | | (hr) | | (mL · hr − 1 · kg − 1) | |
| (Formulation) | Mean | SD | Mean | SD | Mean | SD | Mean | SD | Mean | SD |
| Group 1 | 1,333,194.6 | 197,513.8 | 174,800.0 | 19,942.4 | 2.8 | 1.1 | 3.3 | 0.32 | 68.1 | 10.0 |
| Composition I | | | | | | | | | | |
| Group 2 | 912,679.9 | 161,665.7 | 132,500.0 | 19,710.4 | 4.0 | 0.0 | 3.0 | 0.22 | 100.6 | 18.9 |
| Composition II | | | | | | | | | | |
| Group 3 | 1,480,971.8 | 333,521.8 | 180,600.0 | 34,121.8 | 3.6 | 2.6 | 3.3 | 0.64 | 65.3 | 14.8 |
| Standard | | | | | | | | | | |
| Formulation | | | | | | | | | | |
| Group 4 | 216,542.1 | 125,241.2 | 31,080.0 | 7851.9 | 4.4 | 2.2 | 4.2 | 0.70 | 548.9 | 67.9 |
| Control | | | | | | | | | | |
| Formulation | | | | | | | | | | |

Composition I according to the invention performed exceedingly well. The AUC of Composition I was 1,333, 194.6 hr·ng/mL as compared to an AUC for TRICOR® of 1,480, 971.8 hr·ng/mL—only a 9.9% difference. The Cₘₐₓ for Composition I was 174,800.0 ng/mL, as compared to a Cₘₐₓ of 180, 600.0 ng/mL for TRICOR®—only a 3.2% difference. Most surprising was that Composition I exhibited a Tₘₐₓ less than that for TRICOR®: 2.8 hr as compared to 3.6 hr.

The dose and mean AUC were then used to compute the relative exposure (%) for each Group. “Relative exposure” represents the extent of overall bioavailability, the expression of relative exposure projects how the test and control formulation perform with respect to the standard (which is assigned 100%).

TABLE 3

| Group | | Mean AUC | Relative |
|---|---|---|---|
| (Formulation) | Dose (mg/kg) | (h*ng/mL) | Exposure % |
| Group 1 | 90 | 1333194.6 | 90.0 |
| Composition I | | | |
| Group 2 | 90 | 912679.9 | 61.6 |
| Composition II | | | |
| Group 3 | 90 | 1480971.8 | 100.0 |
| Standard Formulation | | | |
| Group 4 | 90 | 216542.1 | 14.6 |
| Control Formulation | | | |

Again, the results demonstrate the excellent bioavailability of Composition 1 as compared to TRICOR®.

### EXAMPLE 10

The purpose of this example was to prepare compositions according to the invention comprising acyclovir, and then to test the formulations for drug release in a transdermal delivery system.

Acyclovir is a synthetic nucleoside analogue active against herpes viruses. The drug is sold commercially under the trade name ZOVIRAX®. Acyclovir is a white, crystalline powder with the molecular formula C₈H₁₁N₅O₃ and a molecular weight of 225. The maximum solubility in water at 37° C. is 2.5 mg/mL. The pkₐ's of acyclovir are 2.27 and 9.25. The chemical name of acyclovir is 2-amino-1,9-dihydro-9-[(2-hydroxyethoxy)methyl]-6H-purin-6-one; it has the following structural formula: 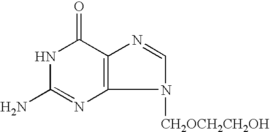
Acyclovir is a synthetic purine nucleoside analogue with in vitro and in vivo inhibitory activity against herpes simplex virus types 1 (HSV-1), 2 (HSV-2), and varicella-zoster virus (VZV). The inhibitory activity of acyclovir is highly selective due to its affinity for the enzyme thymidine kinase (TK) encoded by HSV and VZV. This viral enzyme converts acyclovir into acyclovir monophosphate, a nucleoside analogue. The monophosphate is further converted into diphosphate by cellular guanylate kinase and into triphosphate by a number of cellular enzymes.

A. Acyclovir in N-methyl-pyrrolidinone

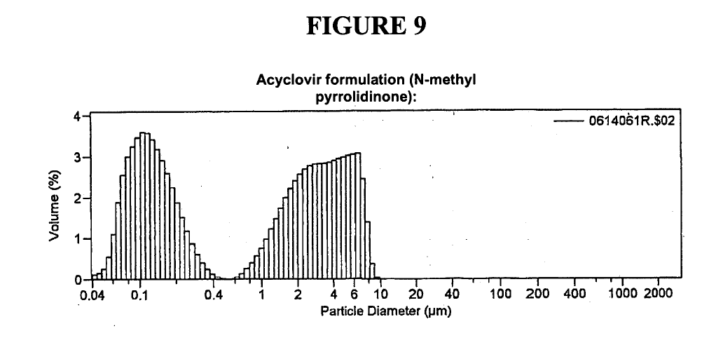

Acyclovir was dissolved in N-methyl-pyrrolidinone. Oil, polysorbate 80, and water (see Table 3) were then added and mixed well with a paddle stirrer. The mixture was then fed into a high pressure homogenizer (APV Invensys, model APV-1000) at 10,000 psi for two passes. The resultant composition, described below in Table 4, comprised acyclovir dissolved in the solvent N-methyl-pyrrolidinone and nanoparticulate acyclovir particles associated with the surface stabilizer, polysorbate 80, present in the water portion of the emulsion. The resultant particle size of acyclovir was measured, as shown in FIG. 9. The particle size distribution showed a bimodal curve, with a significant portion of the acyclovir particles having a size of less than about 0.4 microns, and a second group of particles having a size greater than about 1 micron but less than about 10 microns.

TABLE 4

| Ingredient | Quantity | |
|---|---|---|
| Acyclovir | 5.0 | gm |
| N-methyl-pyrrolidinone | 10 | gm |
| Polysorbate 80 | 9.4 | gm |
| Mineral oil (light) | 47.5 | gm |
| Water | 27.9 | gm |

B. Acyclovir in Ethanol

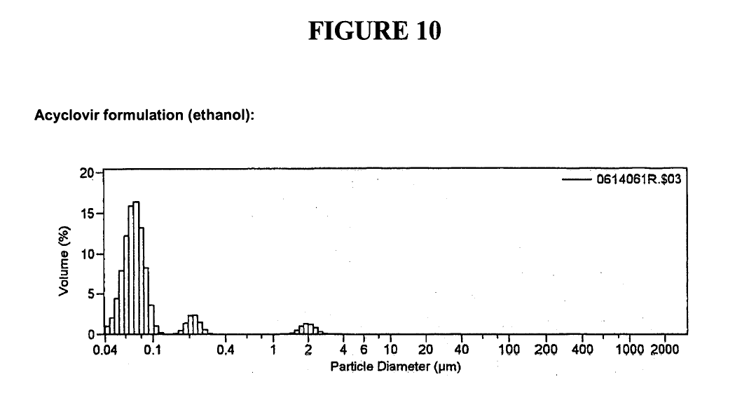

Acyclovir was dispersed in ethanol. Oil, polysorbate 80, and water (Table 5) were then added and mixed well with a paddle stirrer. The mixture was then fed into a high pressure homogenizer (APV Invensys, model APV-1000) at 10,000 psi for two passes. The resultant composition, described below in Table 5, comprised acyclovir dissolved in the solvent ethanol and nanoparticulate acyclovir particles associated with the surface stabilizer polysorbate 80 present in the water portion of the emulsion. The resultant particle size of acyclovir was measured, as shown in FIG. 10. The particle size distribution showed that almost all of the acyclovir particles were less than about 0.1 microns in diameter.

TABLE 5

| Ingredient | Quantity | |
|---|---|---|
| Acyclovir | 5.0 | gm |
| Ethanol | 8.8 | gm |
| Polysorbate 80 | 9.4 | gm |
| Soybean oil | 47.7 | gm |
| Water | 29.2 | gm |

C. Transdermal Dosage Forms Comprising the Acyclovir Formulations

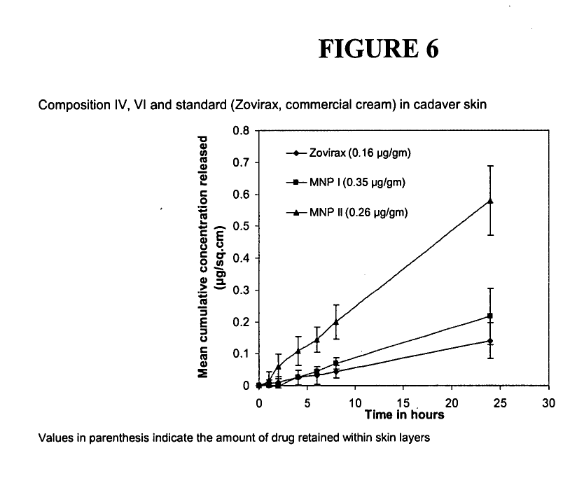

ZOVIRAX® is a commercially available acyclovir topical cream, containing conventional, non-nanoparticulate particles of acyclovir. The rate of release of acyclovir from ZOVIRAX® was compared to the rate of release of acyclovir from the compositions shown in Table 4 (“MNP I”) and Table 5 (“MNP II”). Zovirax® and two formulations prepared above were applied on the known area (1.78 sq. cm) of cadaver skin mounted on a Franz diffusion cell assembly. The amount of acyclovir was the same in all the formulations (5% w/w) and 50 mg of the formulations were applied per skin sample. The amount retained on the skin represents the fraction of the drug that is available for local action, and the amount of API transmitted across the skin indicates the fraction infused into systemic circulation. The results of the comparison, shown in FIG. 6, demonstrate a significant increase in drug release for the formulation of the invention as compared to the prior art, conventional acyclovir formulation.

### EXAMPLE 11

The purpose of this example was to prepare compositions according to the invention comprising cyclosporine.

Cyclosporine is commercially available under the trade names SANDIMMUNE® and NEORAL®. It is a cyclic polypeptide immunosuppressant agent consisting of 11 amino acids. It is produced as a metabolite by the fungus species Beauveria nlyea. Chemically, cyclosporine is designated as [R—[R*,R*-(E)]]-cyclic(L-alanyl-D-alanyl-N-methyl-L-leucyl-N-methyl-L-leucyl-N-methyl-L-valyl-3-hydroxy-N, 4-dimethyl-L-2-amino-6-octenoyl-L-α-amino-butyryl-N-methylglycyl-N-methyl-L-leucyl-L-valyl-N-methyl-L-leucyl). The chemical structure of cyclosporine (also known as cyclosporin A) is:

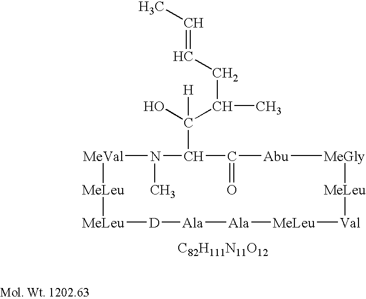

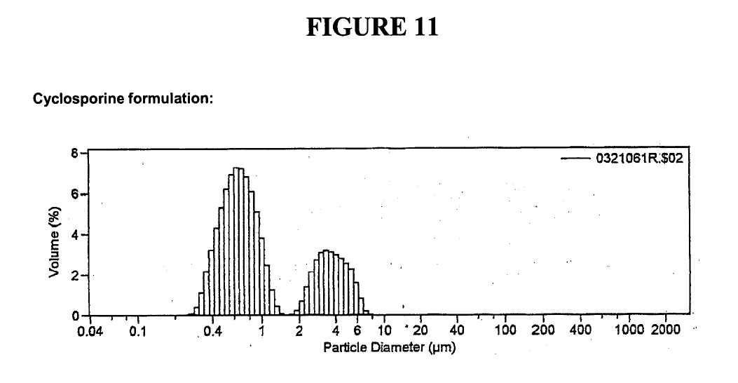

Cyclosporine was dissolved in ethanol. Oil, polysorbate 80, and water (Table 5) were then added and mixed well with a paddle stirrer. The mixture was then fed into a high pressure homogenizer (APV Invensys, model APV-1000) at 10,000 psi for two passes. The resultant composition, described below in Table 6, comprised cyclosporine dissolved in the solvent ethanol and nanoparticulate cyclosporine particles associated with the surface stabilizer polysorbate 80 present in the water portion of the emulsion. The resultant particle size of cyclosporine was measured, as shown in FIG. 11. The particle size distribution showed a bimodal curve, with a significant portion of the cyclosporine particles having a size of less than about 1 micron, and a second group of particles having a size greater than about 2 microns but less than about 8 microns.

TABLE 6

| Ingredient | Quantity | |
|---|---|---|
| Cyclosporine | 5.0 | gm |
| Ethanol | 8.8 | gm |
| Polysorbate 80 | 9.4 | gm |
| Mineral oil (light) | 47.7 | gm |
| Water | 29.2 | gm |

### EXAMPLE 12

The purpose of this example was to prepare compositions according to the invention comprising estradiol, and then to test the formulations for drug release in a transdermal delivery system.

Estradiol is a white crystalline powder, chemically described as estra-1,3,5(10)-triene-3,17β-diol. It has an empirical formula of C₁₈H₂₄O₂ and molecular weight of 272.39. Estradiol is chemically described as estra-1,3,5 (10)-triene-3,17β-diol and has the following structural formula:

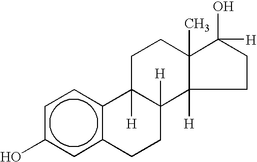

The typical procedure used in each experiment is as follows: dissolve estradiol in ethanol, add the oil (e.g., soybean oil, tricaprylin, or squalane) and polysorbate 80. Add water to the resulting mixture under high-shear mixing (Silverson high-speed mixer) at 9000 rpm. Run for about 3 minutes to obtain an emulsion. Using this process, the following estradiol formulations were prepared.

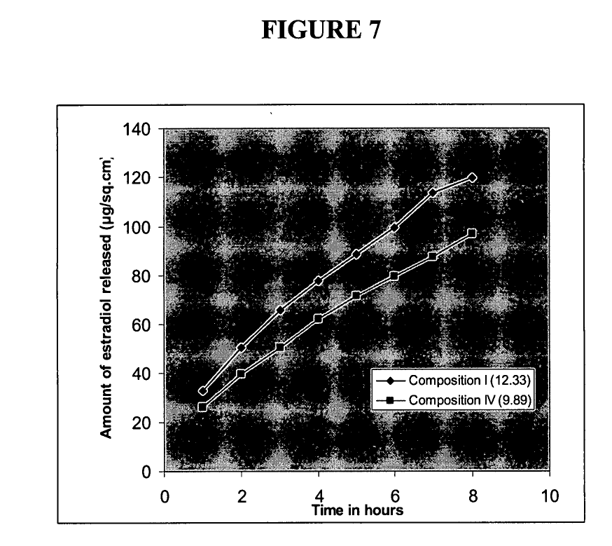

A. Estradiol Formulation #1: Estradiol and Soybean Oil in Ethanol (“Composition I” in FIG. 7)

TABLE 7
Estradiol Formulation #1

| Ingredient | Quantity | |
|---|---|---|
| Estradiol | 0.25 | gm |
| Ethanol | 8.8 | gm |
| Polysorbate 80 | 9.4 | gm |
| Soybean oil | 50.2 | gm |
| Water | 31.35 | gm |

B. Estradiol Formulation #2: Estradiol and Soybean Oil in N-methylpyrrolidinone (“Composition IV” in FIG. 6)

TABLE 8
Estradiol Formulation #2

| Ingredient | Quantity | |
|---|---|---|
| Estradiol | 0.25 | gm |
| N-methyl pyrrolidinone | 8.8 | gm |
| Polysorbate 80 | 9.4 | gm |
| Soybean oil | 50.2 | gm |
| Water | 31.35 | gm |

C. Estradiol Formulation #3: Estradiol and Tricaprylin

TABLE 9
Estradiol Formulation #3

| Ingredient | Quantity | |
|---|---|---|
| Estradiol | 0.25 | gm |
| Ethanol | 8.8 | gm |
| Polysorbate 80 | 9.4 | gm |
| Tricaprylin | 50.2 | gm |
| Water | 31.35 | gm |

D. Estradiol Formulation #4: Estradiol and Squalane

TABLE 19
Estradiol Formulation #4

| Ingredient | Quantity | |
|---|---|---|
| Estradiol | 0.25 | gm |
| Ethanol | 8.8 | gm |
| Polysorbate 80 | 9.4 | gm |
| Squalane | 50.2 | gm |
| Water | 31.35 | gm |

FIG. 1 illustrates the results of in vitro studies of ethanol (Estradiol Formulation #1) versus N-methylpyrrolidinone (Estradiol Formulation #2) on the rate of release of estradiol across an artificial membrane. The values in parentheses in FIG. 7 indicate the flux rate of estradiol across the artificial membrane: 12.33 for Estradiol Formulation #1 and 9.89 for Estradiol Formulation #2.

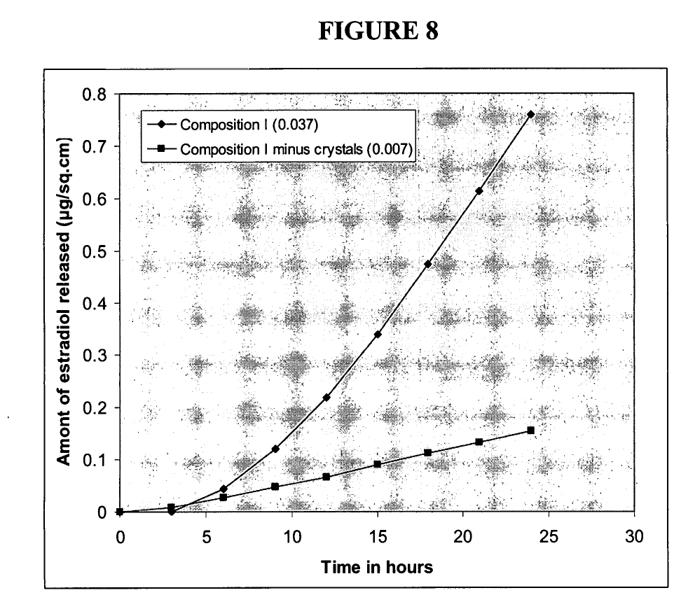

A fraction of estradiol drug was present as solid crystals in Estradiol Formulation #1. The solid fraction was separated by centrifugation to evaluate the contribution and effect of its presence in the formulation on release kinetics of estradiol. The estradiol release profile for Estradiol Formulation #1 comprising solid estradiol particles and lacking such solid estradiol particles were compared. The results are shown in FIG. 8, with the values in parenthesis indicate flux rate of the drug across the skin: Estradiol Formulation #1 (0.037) and Estradiol Formulation #1 lacking drug particles (0.007). Thus, the composition lacking crystalline estradiol particles exhibits a significantly slower rate of release of estradiol, and less hormone is released per microgram per square centimeter of cadaver skin.

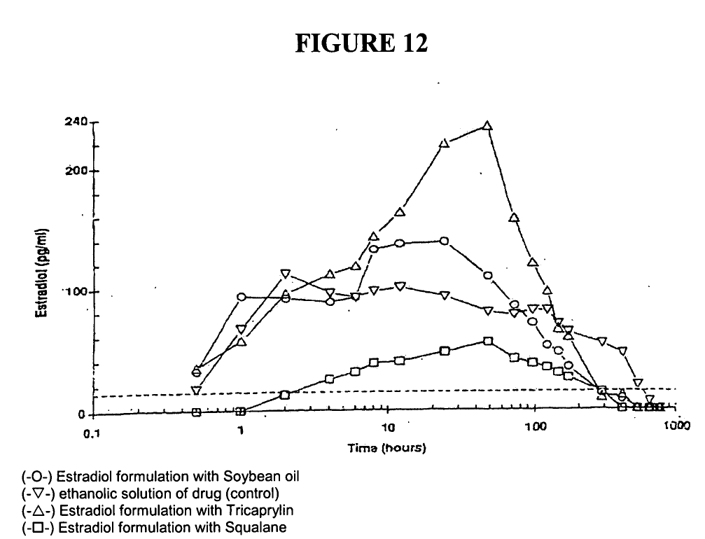

FIG. 12 depicts the in vivo release profile of Estradiol Formulation Nos. 2, 3, and 4 as compared to an ethanolic solution of estradiol, used as a control. Single doses of each formulation (0.42 mL) containing 1 mg of 17 beta estradiol were applied topically to Ovariectomized Rhesus monkeys, with four (4) monkeys per group. Following administration, blood samples were taken from the monkeys at time 0 and at p eriodic intervals following administration: 0 (pre-dose), 0.5, 1, 2, 4, 6, 8, 12, 24, 48, 72, 96, 120, 144, and 168 hr post-dosing. Serum levels of estradiol were then measured in each blood sample (assay of serum estradiol using radioimmunoassay).

The results show that Estradiol Formulation #3, comprising tricaprylin, exhibited the highest rate of drug release, with Estradiol Formulation #2, comprising soybean oil, having the next highest rate. Estradiol Formulation #4, comprising squalane, exhibited the lowest level of drug release.

### EXAMPLE 13

The purpose of this example was to prepare compositions according to the invention comprising amphiphilic drugs, such as cetirizine and nicotine, and then to test the transdermal release profile of the compositions.

A. Cetirizine

Cetirizine HCl is an orally active and selective H₁-receptor antagonist. The chemical name is (±)-[2-[4-[(4-chlorophenyl)phenylmethyl]-1-piperazinyl]ethoxy]acetic acid, dihydrochloride. Cetirizine HCl is a racemic compound with an empirical formula of C₂₁H₂₅ClN₂O₃.2HCl. The molecular weight is 461.82 and the chemical structure is shown below: 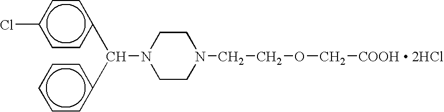
Cetirizine HCl is a white, crystalline powder and is water soluble. The compound is commercially available under the trade name Zyrtec®.

Cetirizine was dissolved in ethanol, and soybean oil and polysorbate 80 were then added to the solution (Table 11). Water was added to the resultant mixture under high-shear mixing (Silverson high-speed mixer) at 9000 rpm. The homogenizer was run for about 3 minutes to obtain an emulsion.

TABLE 11

| Ingredient | Quantity | |
|---|---|---|
| Cetirizine | 0.4 | gm |
| Ethanol | 8.8 | gm |
| Polysorbate 80 | 9.4 | gm |
| Soybean oil | 50.2 | gm |
| Water | 31.3 | gm |

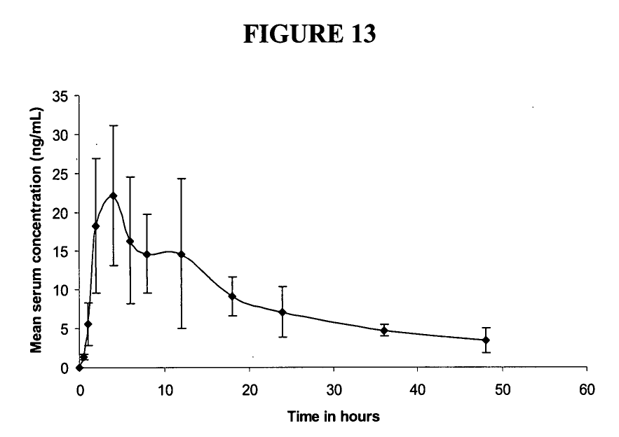

FIG. 13 depicts the in vivo release profile of cetirizine from the formulation shown in Table 10 over time in rabbits. The formulation (2 mL containing 4 mg of cetirizine per gram) was applied topically to three male rabbits. Following administration, blood samples were taken from the rabbits at time 0 and at periodic intervals following administration: 0 (pre-dose), 0.5, 1, 2, 4, 6, 8, 12, 18, 24, 36, 48 hours post-dose. Serum levels of cetirizine were then measured in each blood sample as determined by liquid chromatography-mass spectrometry (LC-MS).

B. Nicotine

Nicotine is a tertiary amine composed of a pyridine and a pyrrolidine ring. It is a colorless to pale yellow, freely water-soluble, strongly alkaline, oily, volatile, hygroscopic liquid obtained from the tobacco plant. Nicotine has a characteristic pungent odor and turns brown on exposure to air or light. Nicotine has the chemical name S-3-(1-methyl-2-pyrrolidinyl) pyridine, the molecular formula C₁₀H₁₄N₂, the molecular weight 162.23, and the following structural formula:

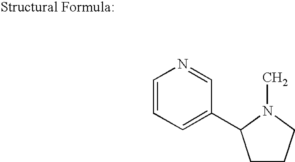

Nicotine was dissolved in ethanol, followed by the addition of squalane, polysorbate 80 and water (Table 12). The composition was mixed well using a paddle stirrer. The composition was then fed into a high-pressure homogenizer (APV Invensys, model APV-1000) and the pressure was tuned to 10,000 psi. The mixture was run through the homogenizer for 2 passes.

TABLE 12

| Ingredient | Quantity | |
|---|---|---|
| Nicotine | 3.0 | gm |
| Ethanol | 2.0 | gm |
| Polysorbate 80 | 9.4 | gm |
| Squalane | 52.2 | gm |
| Water | 33.4 | gm |

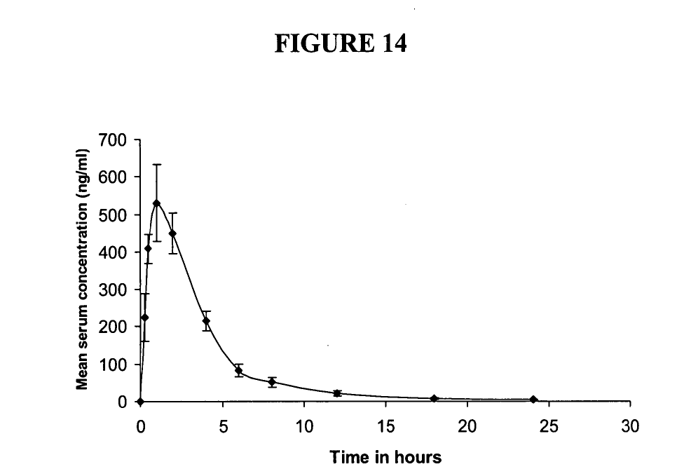

FIG. 14 depicts the in vivo release profile of nicotine from the formulation shown in Table 12 over time in rabbits. The formulation (2 mL containing 30 mg of nicotine per gram of formulation) was applied topically to three male rabbits. Following administration, blood samples were taken from the rabbits at time 0 and at periodic intervals following administration: 0 (pre-dose), 0.5, 1, 2, 4, 6, 8, 12, 18, 24, 36, 48 hours post-dose. Serum levels of nicotine were then measured in each blood sample as determined by liquid chromatography-mass spectrometry (LC-MS).

### EXAMPLE 14

The purpose of this example was to prepare compositions according to the invention comprising hydrophilic drugs, such as naltrexone, alendronic acid, and cetirizine dihydrochloride, and then to test the transdermal release profile of the compositions.

A. Naltrexone

Ethanol, soybean oil and polysorbate 80 were mixed together (Table 12). Naltrexone HCl was then dissolved in water and added to the solvent/oil/stabilizer mixture under high-shear mixing (Silverson high-speed mixer) at 9000 rpm. The homogenizer was run for about 3 minutes to obtain an emulsion. The pH of the resulting composition was then adjusted with citric acid to a pH of 6.76.

TABLE 13

| Ingredient | Quantity |
|---|---|
| Naltrexone HCl | 0.2 gm |
| Ethanol | 8.8 gm |
| Polysorbate 80 | 9.4 gm |
| Soybean oil | 50.2 gm |
| Water | 31.4 gm |
| Citric acid | g.s to pH 6.7 |

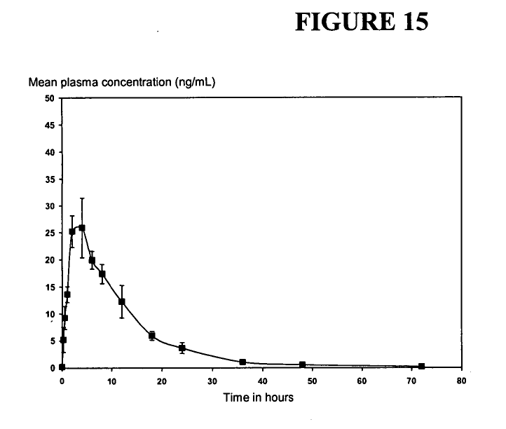

FIG. 15 depicts the in vivo release profile of naltrexone hydrochloride from the formulation shown in Table 13 over time in rabbits. The formulation (2 ml of the formulation containing 10 mg of Naltrexone HCl per gram formulation) was applied topically to three male rabbits. Following administration, blood samples were taken from the rabbits at time 0 and at periodic intervals following administration: 0 (pre-dose), 0.5, 1, 2, 4, 6, 8, 12, 18, 24, 36, 48 hours post-dose. Serum levels of naltrexone hydrochloride were then measured in each blood sample as determined by liquid chromatography-mass spectrometry (LC-MS).

B. Alendronic Acid

Alendronic acid was dispersed in ethanol. Next, polysorbate 80, oil and water were added to the composition (Table 13). The resulting composition was mixed well using a paddle stirrer. The composition was then fed into a high-pressure homogenizer (APV Invensys, model APV-1000), and the pressure was tuned to 10,000 psi. The composition was run through the homogenizer for 2 passes.

TABLE 14

| Ingredient | Quantity | |
|---|---|---|
| Alendronic acid | 1.0 | gm |
| Ethanol | 8.8 | gm |
| Polysorbate 80 | 9.4 | gm |
| Soybean oil | 50.2 | gm |
| Water | 31.7 | gm |

### EXAMPLE 15

### Thermostable Micellar Nanoparticle Compositions

Micellar nanoparticles are quite viscous and cannot be readily sterilized using aseptic filtration devices, such as filtration using a 0.2 micron filter. Terminal heat sterilization, however, is a desirable method for sterilizing such pharmaceutical compositions. A problem is that, typically, micellar nanoparticle formulations are not stable at elevated temperatures, e.g., at temperatures above 50° C., and therefore cannot be readily autoclaved.

The present invention however provides micellar nanoparticle drug compositions which are heat stabile and therefore amenable to heat sterilization.

A. Preparation of Thermostable Compositions

The following compositions were typically made by combining the alcohol and oil and adding that mixture to a thermostable surfactant, such as a Pluronic, dissolved in water under high-shear mixing, e.g., via a Silverson high-speed mixer at 9000 rpm for about 3 minutes to obtain an emulsion.

Composition A

TABLE 15

| Ingredient | Quantity | |
|---|---|---|
| Ethanol | 8.8 | gm |
| Pluronic ® F-68 | 9.4 | gm |
| Soybean oil | 50.2 | gm |
| Water | 31.7 | gm |

Ethanol and soybean oil were mixed together (Table 15). Next, the Pluronic® F-68 was dissolved in water. The ethanol mixture and Pluronic F-68 solution were added together under high-shear mixing (Silverson high-speed mixer) at 9000 rpm. The homogenizer was run for about 3 minutes to obtain an emulsion.

Composition B

TABLE 16

| Ingredient | Quantity | |
|---|---|---|
| Ethanol | 8.8 | gm |
| Pemulen TR-2 | 0.25 | gm |
| Soybean oil | 50.2 | gm |
| Water | 41.1 | gm |

The ethanol and soybean oil were mixed together (Table 16). Pemulen TR-2 was dispersed in the water. The ethanol mixture and the Pemulen TR-2 dispersion were combined under high-shear mixing (Silverson high-speed mixer) at 9000 rpm. The homogenizer was run for about 3 minutes to obtain an emulsion.

Composition C

TABLE 17

| Ingredient | Quantity | |
|---|---|---|
| Ethanol | 8.8 | gm |
| Cremophor RH-40 | 9.4 | gm |
| Soybean oil | 50.2 | gm |
| Water | 31.7 | gm |

The ethanol, Cremophor RH-40 and soybean oil were mixed together (Table 17). The water was added under high-shear mixing (Silverson high-speed mixer) at 9000 rpm. The homogenizer was run for about 3 minutes to obtain an emulsion.

Composition D

TABLE 18

| Ingredient | Quantity | |
|---|---|---|
| N-methyl-pyrrolidinone | 8.8 | gm |
| Pluronic ® F-68 | 9.4 | gm |
| Mineral oil (light) | 50.2 | gm |
| Water | 31.7 | gm |

The N-methyl-pyrrolidinone and mineral oil were mixed together (Table 18). The Pluronic F-68 was dissolved in water. The mineral oil mixture and Pluronic F-68 solution were combined under high-shear mixing (Silverson high-speed mixer) at 9000 rpm. The homogenizer was run for about 3 minutes to obtain an emulsion.

Composition E

TABLE 19

| Ingredient | Quantity | |
|---|---|---|
| Ethanol | 8.8 | gm |
| Span 80 | 9.4 | gm |
| Soybean oil | 50.2 | gm |
| Water | 31.7 | gm |

The ethanol, Span 80 and soybean oil were mixed together (Table 19). The water was added under high-shear mixing (Silverson high-speed mixer) at 9000 rpm. The homogenizer was run for about 3 minutes to obtain an emulsion.

The following formulations were prepared as described above for Compositions A-E.

TABLE 20
Composition F

| Ingredient | Quantity | |
|---|---|---|
| Ethanol | 8.8 | gm |
| Arlacel | 9.4 | gm |
| Soybean oil | 50.2 | gm |
| Water | 31.7 | gm |

TABLE 21
Composition G

| Ingredient | Quantity | |
|---|---|---|
| Ethanol | 8.8 | gm |
| PEG-20 stearate | 9.4 | gm |
| Soybean oil | 50.2 | gm |
| Water | 31.7 | gm |

TABLE 22
Composition H

| Ingredient | Quantity | |
|---|---|---|
| Ethanol | 8.8 | gm |
| Pluronic ® F-68 | 0.9 | gm |
| Soybean oil | 50.2 | gm |
| Water | 40.1 | gm |

TABLE 23
Composition I

| Ingredient | Quantity | |
|---|---|---|
| Ethanol | 8.8 | gm |
| Pluronic ® F-68 | 9.4 | gm |
| Soybean oil | 50.2 | gm |
| Water | 31.7 | gm |

TABLE 24
Composition J

| Ingredient | Quantity | |
|---|---|---|
| Ethanol | 8.8 | gm |
| Pluroni ® F-127 | 0.9 | gm |
| Soybean oil | 50.2 | gm |
| Water | 40.1 | gm |

TABLE 25
Composition K

| Ingredient | Quantity | |
|---|---|---|
| Ethanol | 8.8 | gm |
| Brij ® 93 | 9.4 | gm |
| Soybean oil | 50.2 | gm |
| Water | 31.7 | gm |

TABLE 26
Composition L

| Ingredient | Quantity | |
|---|---|---|
| Ethanol | 8.8 | gm |
| Polysorbate 80 | 9.4 | gm |
| Soybean oil | 50.2 | gm |
| Water | 31.7 | gm |

B. Thermal Challenge

Sample amounts of the prepared emulsion of Compositions A-L were poured into glass vials and then autoclaved at 120° C. at 15 psi pressure for 25 minutes. The droplet size was measured before and after autoclaving. The percentage change served as indicator, along with visible observation, of whether the emulsion was stable after heat sterilization. Hence, “no change” indicates that the emulsion was not detrimentally affected by heat sterilization.

TABLE 27

| | | Effect of | |
|---|---|---|---|
| | | Autoclaving | Mean droplet size |
| | | (Physical | before-after (% |
| No. | Formulation | Appearance) | change) |
| 1. Composition E | MNP with Span 80 | No change | 0.830-0.840 (+1.2%) |
| | (9.4% w/w) | | |
| 2. Composition F | MNP with Arlacel | No change | 1.626-1.624 (−0.123%) |
| | (9.4% w/w) | | |
| 3. Composition G | MNP with PEG-stearate | Phase separation | |
| | (9.4% w/w) | | |
| 4. Composition H | MNP with Pluronic F68 | No change | 0.867-0.831 (−4.15%) |
| | (0.9% w/w) | | |
| 5. Composition I | MNP with Pluronic F68 | No change | 0.469-0.510 (+4.1%) |
| | (9.4% w/w) | | |
| 6. Composition J | MNP with Pluronic | No change | ND |
| | F127 (0.9% w/w) | | |
| 7. Composition B | MNP with Pemulen TR- | No change | 0.721-0.717 (−0.4%) |
| | 2 (0.25% w/w) | | |
| 8. Composition C | MNP with Cremophore | No change | 1.006-1.180 (+17.4%) |
| | RH4O (9.4% w/w) | | |
| 9. Composition K | MNP with Brij | Phase separation | |
| | (9.4% w/w) | | |
| 10. Composition | MNP with PS 80 (9.4% | Phase separation | |
| L | w/w) (CONTROL) | | |

The results shown in Table 27 demonstrate the dramatic and unexpected thermostability of the compositions of the invention.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A method for preparing a pharmaceutical dosage form comprising:
(a) forming an emulsion base by suspending an active pharmaceutical ingredient (API) in a mixture of oil, solvent, stabilizer, and water or buffer to form an emulsion base, wherein:
(i) the active pharmaceutical ingredient is poorly soluble in the oil, solvent, and water, or
(ii) the active pharmaceutical ingredient is soluble in either or both of oil and solvent, but is not soluble, or is poorly soluble, in water,
(b) homogenizing the emulsion base to form particles of the active pharmaceutical ingredient, droplets comprising solubilized API, or a combination thereof.

2. The method of claim 1, wherein the resultant composition is a mixture of API particles suspended in the emulsion droplets and sterically stabilized particulate API in the water or buffer.

3. The method of claim 1, wherein the active pharmaceutical ingredient is selected from the group consisting of fenofibrate, estradiol, alendronic acid, acyclovir, paclitaxel, and cyclosporine.

4. The method of claim 1, wherein the oil is selected from the group consisting of almond oil (sweet), apricot seed oil, borage oil, canola oil, coconut oil, corn oil, cotton seed oil, fish oil, jojoba bean oil, lard oil, linseed oil (boiled), Macadamia nut oil, medium chain triglycerides, mineral oil, olive oil, peanut oil, safflower oil, sesame oil, soybean oil, squalene, sunflower seed oil, tricaprylin (1,2,3-trioctanoyl glycerol), and wheat germ oil.

5. The method of claim 1, wherein the solvent is selected from the group consisting of isopropyl myristate, triacetin, N-methylpyrrolidinone, aliphatic and aromatic alcohols, ethanol dimethyl sulfoxide, dimethyl acetamide, ethoxydiglycol, polyethylene glycols, and propylene glycol.

6. The method of claim 1, wherein the stabilizer is selected from the group consisting of sorbitan esters, glycerol esters, polyethylene glycol esters, block polymers, acrylic polymers (such as Pemulen), ethoxylated fatty esters (such as Cremophor RH-40), ethoxylated alcohols (such as Brij), ethoxylated fatty acids (such as Tween), monoglycerides, silicon based surfactants, and polysorbates.

7. The method of claim 6, wherein the sorbitan ester stabilizer is Span and Arlacel, wherein the glycerol ester is glycerin monostearate, wherein the polyethylene glycol ester is polyethylene glycol stearate, wherein the block polymer is a Pluronic, wherein the acrylic polymer is Pemulen, wherein the ethoxylated fatty ester is Cremophor RH-40, wherein the ethoxylated alcohol is Brij, and wherein the ethoxylated fatty acid is Tween 20.

8. The method of claim 1, wherein the homogenizing step is performed via a high-pressure system at 1,000 to 40,000 psi.

9. The method of claim 1, wherein the resultant active pharmaceutical ingredient particles (API), droplets comprising solubilized API, or a combination thereof, have an average or mean particle size selected from the group consisting of less than about 10 microns, less than about 9 microns, less than about 8 microns, less than about 7 microns, less than about 6 microns, less than about 5 microns, less than about 4 microns, less than about 3 microns, less than about 2 microns, and about 1 micron or greater.

10. The method of claim 9, wherein the resultant active pharmaceutical ingredient particles (API), droplets comprising solubilized API, or a combination thereof, have a mean particle size selected from the group consisting of less than about 1 micron, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 mu.

11. A method for preparing fenofibrate particles comprising
(a) dissolving fenofibrate in N-methyl-pyrrolidinone to form a solution,
(b) adding medium chain triglyceride to the fenofibrate solution,
(c) adding Pluronic dissolved in water to the solution, and
(d) subjecting the solution to high-pressure homogenization to produce fenofibrate particles.

12. A method of preparing a transdermal dosage form comprising:
(a) dissolving an active pharmaceutical ingredient (API) in a mixture of (i) at least one oil, (ii) at least one solvent, and (iii) at least one stabilizer to form an emulsion pre-mix,
(b) adding water or buffer to the emulsion pre-mix, and
(c) homogenizing or vigorously stirring the mixture,
whereby the API is precipitated into particles.

13. The method of claim 12, wherein the API is selected from the group consisting of acyclovir, cyclosporine, naltrexone, alendronic acid, cetirizine, nicotine, testosterone, progesterone, or estradiol.

14. A pharmaceutical dosage form comprising:
(a) at least one active pharmaceutical ingredient, wherein the active pharmaceutical ingredient is in a solid particulate state and in a soluble state,
(b) at least one solvent,
(c) at least one oil,
(d) at least one surfactant, and
(e) water.

15. The pharmaceutical dosage form of claim 14, wherein the active pharmaceutical ingredient is selected from the group consisting of fenofibrate, alendronic acid, acyclovir, paclitaxel, cyclosporine, naltrexone, cetirizine, nicotine, testosterone, progesterone, and estradiol.

16. The pharmaceutical dosage form of claim 14, wherein the composition comprises globules of oil comprising dissolved active pharmaceutical ingredient, wherein the globules have a diameter of less than about 10 microns.

17. The pharmaceutical dosage form of claim 16, wherein the globules having a diameter selected from the group consisting of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

18. The pharmaceutical dosage form of claim 14, which is a transdermal dosage form.

19. A method of treating a subject in need comprising applying the transdermal dosage form of claim 18 to the skin of the subject.

20. The method of claim 19, wherein the transdermal dosage form is applied as a topical cream onto the skin of the subject.
